SEQUENCE LISTING (11 sequences)

SEQ ID NO: 1 - protein, 249 aa, Homo Sapiens
MKLNISFPATGCQKLIEVDDERKLRTFYEKRMATEVAADALGEEWKGYVVRISGGNDKQGFPMKQGVLTHGRVRLLLSKGHSCYRPRRTGERKRKSVRGCIVDANLSVLNLVIVKKGEKDIPGLTDTTVPRRLGPKRASRIRKLFNLSKEDDVRQYVVRKPLNKEGKKPRTKAPKIQRLVTPRVLQHKRRRIALKKQRTKKNKEEAAEYAKLLAKRMKEAKEKRQEQIAKRRRLSSLRASTSKSESSQK

SEQ ID NO: 2 - protein, 2549 aa, Homo Sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 3 - protein, 655 aa, Homo Sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 4 - protein, 480 aa, Homo Sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASSTA

SEQ ID NO: 5 - protein, 403 aa, Homo Sapiens [sequence omitted]

SEQ ID NO: 6 - protein, 673 aa, Homo Sapiens
MAEAPASPAPLSPLEVELDPEFEPQSRPRSCTWPLQRPELQASPAKPSGETAADSMIPEEEDDEDDEDGGGRAGSAMAIGGGGGSGTLGSGLLLEDSARVLAPGGQDPGSGPATAAGGLSGGTQALLQPQQPLPPPQPGAAGGSGQPRKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNSNKYTKSRGRAAKKKAALQTAPESADDSPSQLSKWPGSPTSRSSDELDAWTDFRSRTNSNASTVSGRLSPIMASTELDEVQDDDAPLSPMLYSSSASLSPSVSKPCTVELPRLTDMAGTMNLNDGLTENLMDDLLDNITLPPSQPSPTGGLMQRSSSFPYTTKGSGLGSPTSSFNSTVFGPSSLNSLRQSPMQTIQENKPATFSSMSHYGNQTLQDLLTSDSLSHSDVMMTQSDPLMSQASTAVSAQNSRRNVMLRNDPMMSFAAQPNQGSLVNQNLLHHQHQTQGALGGSRALSNSVSNMGLSESSSLGSAKHQQQSPVSQSMQTLSDSLSGSSLYSTSANLPVMGHEKFPSDLDLDMFNGSLECDMESIIRSELMDADGLDFNFDSLISTQNVVGLNVGNFTGAKQASSQSWVPG

SEQ ID NO: 7 - protein, 1210 aa, Homo Sapiens [sequence omitted]

SEQ ID NO: 8 - protein, 379 aa, Homo Sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 9 - protein, 3256 aa, Homo Sapiens
MWPTRRLVTIKRSGVDGPHFPLSLSTCLFGRGIECDIRIQLPVVSKQHCKIEIHEQEAILHNFSSTNPTQVNGSVIDEPVRLKHGDVITIIDRSFRYENESLQNGRKSTEFPRKIREQEPARRVSRSSFSSDPDEKAQDSKAYSKITEGKVSGNPQVHIKNVKEDSTADDSKDSVAQGTTNVHSSEHAGRNGRNAADPISGDFKEISSVKLVSRYGELKSVPTTQCLDNSKKNESPFWKLYESVKKELDVKSQKENVLQYCRKSGLQTDYATEKESADGLQGETQLLVSRKSRPKSGGSGHAVAEPASPEQELDQNKGKGRDVESVQTPSKAVGASFPLYEPAKMKTPVQYSQQQNSPQKHKNKDLYTTGRRESVNLGKSEGFKAGDKTLTPRKLSTRNRTPAKVEDAADSATKPENLSSKTRGSIPTDVEVLPTETEIHNEPFLTLWLTQVERKIQKDSLSKPEKLGTTAGQMCSGLPGLSSVDINNFGDSINESEGIPLKRRRVSFGGHLRPELFDENLPPNTPLKRGEAPTKRKSLVMHTPPVLKKIIKEQPQPSGKQESGSEIHVEVKAQSLVISPPAPSPRKTPVASDQRRRSCKTAPASSSKSQTEVPKRGGERVATCLQKRVSISRSQHDILQMICSKRRSGASEANLIVAKSWADVVKLGAKQTQTKVIKHGPQRSMNKRQRRPATPKKPVGEVHSQFSTGHANSPCTIIIGKAHTEKVHVPARPYRVLNNFISNQKMDFKEDLSGIAEMFKTPVKEQPQLTSTCHIAISNSENLLGKQFQGTDSGEEPLLPTSESFGGNVFFSAQNAAKQPSDKCSASPPLRRQCIRENGNVAKTPRNTYKMTSLETKTSDTETEPSKTVSTVNRSGRSTEFRNIQKLPVESKSEETNTEIVECILKRGQKATLLQQRREGEMKEIERPFETYKENIELKENDEKMKAMKRSRTWGQKCAPMSDLTDLKSLPDTELMKDTARGQNLLQTQDHAKAPKSEKGKITKMPCQSLQPEPINTPTHTKQQLKASLGKVGVKEELLAVGKFTRTSGETTHTHREPAGDGKSIRTFKESPKQILDPAARVTGMKKWPRTPKEEAQSLEDLAGFKELFQTPGPSEESMTDEKTTKIACKSPPPESVDTPTSTKQWPKRSLRKADVEEEFLALRKLTPSAGKAMLTPKPAGGDEKDIKAFMGTPVQKLDLAGTLPGSKRQLQTPKEKAQALEDLAGFKELFQTPGHTEELVAAGKTTKIPCDSPQSDPVDTPTSTKQRPKRSIRKADVEGELLACRNLMPSAGKAMHTPKPSVGEEKDIIIFVGTPVQKLDLTENLTGSKRRPQTPKEEAQALEDLTGFKELFQTPGHTEEAVAAGKTTKMPCESSPPESADTPTSTRRQPKTPLEKRDVQKELSALKKLTQTSGETTHTDKVPGGEDKSINAFRETAKQKLDPAASVTGSKRHPKTKEKAQPLEDLAGWKELFQTPVCTDKPTTHEKTTKIACRSQPDPVDTPTSSKPQSKRSLRKVDVEEEFFALRKRTPSAGKAMHTPKPAVSGEKNIYAFMGTPVQKLDLTENLTGSKRRLQTPKEKAQALEDLAGFKELFQTRGHTEESMTNDKTAKVACKSSQPDLDKNPASSKRRLKTSLGKVGVKEELLAVGKLTQTSGETTHTHTEPTGDGKSMKAFMESPKQILDSAASLTGSKRQLRTPKGKSEVPEDLAGFIELFQTPSHTKESMTNEKTTKVSYRASQPDLVDTPTSSKPQPKRSLRKADTEEEFLAFRKQTPSAGKAMHTPKPAVGEEKDINTFLGTPVQKLDQPGNLPGSNRRLQTRKEKAQALEELTGFRELFQTPCTDNPTADEKTTKKILCKSPQSDPADTPTNTKQRPKRSLKKADVEEEFLAFRKLTPSAGKAMHTPKAAVGEEKDINTFVGTPVEKLDLLGNLPGSKRRPQTPKEKAKALEDLAGFKELFQTPGHTEESMTDDKITEVSCKSPQPDPVKTPTSSKQRLKISLGKVGVKEEVLPVGKLTQTSGKTTQTHRETAGDGKSIKAFKESAKQMLDPANYGTGMERWPRTPKEEAQSLEDLAGFKELFQTPDHTEESTTDDKTTKIACKSPPPESMDTPTSTRRRPKTPLGKRDIVEELSALKQLTQTTHTDKVPGDEDKGINVFRETAKQKLDPAASVTGSKRQPRTPKGKAQPLEDLAGLKELFQTPVCTDKPTTHEKTTKIACRSPQPDPVGTPTIFKPQSKRSLRKADVEEESLALRKRTPSVGKAMDTPKPAGGDEKDMKAFMGTPVQKLDLPGNLPGSKRWPQTPKEKAQALEDLAGFKELFQTPGTDKPTTDEKTTKIACKSPQPDPVDTPASTKQRPKRNLRKADVEEEFLALRKRTPSAGKAMDTPKPAVSDEKNINTFVETPVQKLDLLGNLPGSKRQPQTPKEKAEALEDLVGFKELFQTPGHTEESMTDDKITEVSCKSPQPESFKTSRSSKQRLKIPLVKVDMKEEPLAVSKLTRTSGETTQTHTEPTGDSKSIKAFKESPKQILDPAASVTGSRRQLRTRKEKARALEDLVDFKELFSAPGHTEESMTIDKNTKIPCKSPPPELTDTATSTKRCPKTRPRKEVKEELSAVERLTQTSGQSTHTHKEPASGDEGIKVLKQRAKKKPNPVEEEPSRRRPRAPKEKAQPLEDLAGFTELSETSGHTQESLTAGKATKIPCESPPLEVVDTTASTKRHLRTRVQKVQVKEEPSAVKFTQTSGETTDADKEPAGEDKGIKALKESAKQTPAPAASVTGSRRRPRAPRESAQAIEDLAGFKDPAAGHTEESMTDDKTTKIPCKSSPELEDTATSSKRRPRTRAQKVEVKEELLAVGKLTQTSGETTHTDKEPVGEGKGTKAFKQPAKRNVDAEDVIGSRRQPRAPKEKAQPLEDLASFQELSQTPGHTEELANGAADSFTSAPKQTPDSGKPLKISRRVLRAPKVEPVGDVVSTRDPVKSQSKSNTSLPPLPFKRGGGKDGSVTGTKRLRCMPAPEEIVEELPASKKQRVAPRARGKSSEPVVIMKRSLRTSAKRIEPAEELNSNDMKTNKEEHKLQDSVPENKGISLRSRRQDKTEAEQQITEVFVLAERIEINRNEKKPMKTSPEMDIQNPDDGARKPIPRDKVTENKRCLRSARQNESSQPKVAEESGGQKSAKVLMQNQKGKGEAGNSDSMCLRSRKTKSQPAASTLESKSVQRVTRSVKRCAENPKKAEDNVCVKKITTRSHRDSEDI

SEQ ID NO: 10 - protein, 136 aa, Homo Sapiens
MARTKQTARKSTGGKAPRKQLATKAARKSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 11 - protein, 277 aa, Homo Sapiens
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVDADFLYAYSTAPGYYSWRNSKDGSWFIQSLCAMLKQYADKLEFMHILTRVNRKVATEFESFSFDATFHAKKQIPCIVSMLTKELYFYH

(19) United States
(12) Patent Application Publication
(10) Pub. No.: US 2008/0299589 A1
(43) Pub. Date: Dec. 4, 2008

(54) Methods and Materials for Examining the P13k/Akt Pathway in Gliomas and Prostate Cancers

(75) Inventors: Charles L. Sawyers, New York, NY (US); George V. Thomas, Los Angeles, CA (US)

Correspondence Address: GATES & COOPER LLP;HOWARD HUGHES CENTER, 6701 CENTER DRIVE WEST, SUITE 1050, LOS ANGELES, CA, 90045, US

(73) Assignee: The Regents of the University of California, Oakland, CA (US)

(21) Appl. No.: 11/667,430

(22) PCT Filed: Nov. 8, 2004
(86) PCT No.: PCT/US04/37288

§ 371 Date: Mar. 28, 2008

(30) Foreign Application Priority Data
May 11, 2003 (US) 10701490

(51) Int. Cl. G01N 33/574; C12Q 1/02

(52) U.S. Cl. 435/7.23; 435/29

(57) ABSTRACT

The invention disclosed herein provides methods for the examination and/or quantification of the PI3K/Akt and Ras/MAPK biochemical pathways that are disregulated in pathologies such as cancers of the prostate and gliomas. The invention disclosed herein further provides reagents and kits adapted for performing such methods.

### RELATED APPLICATIONS

This application is a continuation-in-part of U.S. patent application Ser. No. 10/701,490 filed Nov. 5, 2003, which claims the benefit of U.S. Provisional Application Ser. No. 60/423,777 filed Nov. 5, 2002, the contents of each of which are incorporated herein by reference.

### STATEMENT OF GOVERNMENT SUPPORT

This invention was made with support from U01 CA88127 from the National Cancer Institute/NIH and K08NS43147-01 from the National Institute of Neurological Disorders and Stroke/NIH. The government may have certain rights to this invention.

### FIELD OF THE INVENTION

The present invention provides methods for the examination of biochemical pathways that are shown to be disregulated in pathologies such as cancer and to reagents adapted for performing these methods.

### BACKGROUND OF THE INVENTION

Cancers are the second most prevalent cause of death in the United States, causing 450,000 deaths per year. One in three Americans will develop cancer, and one in five will die of cancer. While substantial progress has been made in identifying some of the likely environmental and hereditary causes of cancer, there is a need for additional diagnostic and therapeutic modalities that target cancer and related diseases and disorders. In particular, there is for a need a greater understanding of the various biochemical pathways that are involved in disregulated cell growth such as cancer as this will allow for the development of improved diagnostic and therapeutic methods for identifying and treating pathological syndromes associated with such growth disregulation.

Biochemical pathways that are of particular interest in pathologies such as cancer are the PI3K/Akt and Ras/MAPK pathways. Specifically, deregulation of the PI3K/Akt and Ras/MAPK pathways occurs in many types of cancer (see, e.g., Vivanco et al., Nat Rev Cancer. 2: 489-501, 2002), including glioblastoma (GBM) and cancer of the prostate (see, e.g., Vivanco et al., Nat Rev Cancer. 2: 489-501, 2002; Feldkamp et al., Journal of Neurooncology 35: 223-248, 1997; Mischel et al., Brain Pathology, January; 13(1):52-61 2003). Because constitutively activated signal transduction cascades directly modulate biological behavior, and because new molecular approaches to cancer therapy focus on inhibiting these pathways (see, e.g., Sawyers et al., Curr Opin Genet Dev. 12: 111-5, 2002; Drunker et al., Cancer Cell. 1: 31-6, 2002; Kilic et al., Cancer Res. 60: 5143-50, 2000; Neshat et al., Proc Natl Acad Sci USA. 98: 10314-9, 2001), it is critical that they be detected in patient biopsies. Traditionally, biochemical approaches such as Western blots and in vitro kinase assays have been required to assess activation of these pathways (see, e.g., Neshat et al., Proc Natl Acad Sci USA. 98: 10314-9, 2001; Ermoian et al., Clin Cancer Res. 8: 1100-6, 2002). However, these techniques are not feasible on routinely processed tissues such as formalin-fixed, paraffin-embedded patient biopsy samples. Currently, the tools to identify activation pathways in patient biopsy material have not been fully developed. Development of such tools is critical to determine whether these pathway activations have prognostic significance, and to help stratify patients for targeted molecular therapy.

Glioblastoma multiforme (GBM), the most common malignant brain tumor of adults (and one of the most lethal of all cancers) is highly suited for this approach. GBMs have a set of defined molecular lesions with resultant signaling pathway disruptions. The tumor suppressor gene PTEN is altered in 30-40% of GBMs (see, e.g., Liu et al., Cancer Res. 57: 5254-7, 1997; Schmidt et al., J Neuropathol Exp Neurol. 58: 1170-83, 1999; Smith et al., J Natl Cancer Inst. 93: 1246-56, 2001). Since the PTEN lipid phosphatase activity negatively regulates activation of the Akt pathway and its downstream effectors mTOR, FKHR and S6 (see, e.g., Vivanco et al., Nat Rev Cancer. 2: 489-501, 2002), it is possible that PTEN protein deficient GBMs would show coordinated activation of this pathway. Primary GBMs (those that arise as de novo grade IV tumors) also commonly over-express the oncogene EGFR, and its variant EGFRvIII, which activate signaling through both the RAS/MAPK and PI3K/Akt pathways. Therefore, it is also possible that EGFR and EGFRvIII expressing GBMs would show coordinate activation of the ERK and the Akt pathways. To date however, the relationship between these various pathways has not been delineated.

Inactivation of the PTEN tumor suppressor gene is also implicated in the development of both primary and metastatic prostate cancers. Conditional deletions of PTEN in the mouse prostate leads to cancer and many human prostate cancer cell lines and xenografts have PTEN deletions or point mutations (see, e.g. Wang et al., Cancer Cell, 4: 209-221, 2003; Trotman et al., PLoS Biol, 1: E59, 2003; Steck et al., Nat Genet, 15: 356-362, 1997; Pesche et al., Oncogene, 16: 2879-2883, 1998; and Teng et al., Cancer Res, 57: 5221-5225, 1997). The best characterized function of PTEN is as a lipid phosphatase that counteracts the growth and survival promoting effects of phosphatidyl inositol 3-kinase (PI3K). PI3K is a lipid kinase that phosphorylates phosphatidylinositols at the 3-position (PtdIns [3, 4, 5] P₃), which subsequently recruit kinases such as AKT (a potent oncogenic survival factor), leading to a cascade of constitutive activation of downstream effectors (FIG. 4), including the mammalian Target Of Rapamycin (mTOR) (Vivanco et al., Nat Rev Cancer, 2: 489-501, 2002; Hidalgo et al., Oncogene, 19: 6680-6686, 2000; Sawyers et al., Cancer Cell, 4: 343-348, 2003; Fingar et al., Oncogene, 23: 3151-3171, 2004; Luo et al., Cancer Cell, 4: 257-262, 2003).

While researchers have identified a variety of genes and pathways involved in pathologies such as cancer, there is need in the art for additional tools to facilitate the analyses of the regulatory processes that are involved in disregulated cell growth. Moreover, an understanding of how the products of genes involved in disregulated cell growth interact in a larger context is needed for the development of improved diagnostic and therapeutic methods for identifying and treating pathological syndromes associated with growth disregulation. In particular, there remains a need to identify signal transduction events driving oncogenesis and to identify markers useful for assessing progression or inhibition of the oncogenic phenotype. The methods and reagents disclosed herein satisfy this need.

### SUMMARY OF THE INVENTION

Deregulated activation of the PI3K/Akt pathway is common in a variety of different cancers including glioblastomas and cancers of the prostate, bile duct, bladder, breast, colon, endometrium, blood, liver, lung, skin, ovary, pancreas and thyroid to name a few. Consequently, the assessment of this pathway can be used to obtain diagnostic, prognostic and therapeutic information, for example to stratify patients for targeted kinase inhibitor therapy. The disclosure provided herein identifies a series of biomarkers that are associated with deregulated activation of the PI3K/Akt pathway as well as optimized methods for examining these markers. Significantly, the disclosed methods for examining these markers are useful with a wide variety of tissue samples including formalin fixed, paraffin embedded biopsy samples.

As disclosed herein, a series of PI3K/Akt pathway biomarkers associated with cancers such as prostate cancer and glioblastoma multiforme can be examined using for example a series of antibodies such as phospho-specific antibodies. In typical methods, a mammalian cell such as a cancer cell derived from a biopsy sample can be examined for evidence of PI3K/Akt pathway activation by examining a tissue sample containing this cell for the presence of: a phosphorylated S6 polypeptide (SEQ ID NO: 1); a phosphorylated mTOR polypeptide (SEQ ID NO: 2); a phosphorylated FKHR polypeptide (SEQ ID NO: 3); a phosphorylated AKT polypeptide (SEQ ID NO: 4); a phosphorylated ERK polypeptide (SEQ ID NO: 8); or decreased levels of expression of the PTEN polypeptide (SEQ ID NO: 5), wherein the presence of a phosphorylated S6, mTOR, FKHR, AKT or ERK polypeptide, or decreased levels of expression of the PTEN polypeptide, provides evidence of Akt pathway activation in the cell. Optionally the cell is examined for the presence of a plurality of characteristics such as a phosphorylated S6 polypeptide (SEQ ID NO: 1) and decreased levels of expression of the PTEN polypeptide (SEQ ID NO: 5). Certain embodiments of the invention comprise further methodological steps, the step of using the results of the examination to identify and/or assess a therapeutic agent that may be used to treat the cancer such as the step of using the results of the examination to evaluate the effect of an mTOR inhibitor such as rapamycin or an analogue thereof or an EGFR inhibitor such as ZD-1839 or an analogue thereof on a cancer cell.

One preferred embodiment of the invention is a method for identifying a mammalian glioma tumor likely to respond or responsive to an EGFR polypeptide (SEQ ID NO: 7) inhibitor or an mTOR polypeptide (SEQ ID NO: 2) inhibitor, the method comprising examining a sample obtained from the tumor for: the expression of PTEN polypeptide (SEQ ID NO: 5); and the presence of at least one of, a phosphorylated S6 ribosomal polypeptide (SEQ ID NO: 1); a EGFR polypeptide (SEQ ID NO: 7); a phosphorylated AKT polypeptide (SEQ ID NO: 4); and a phosphorylated ERK polypeptide (SEQ ID NO: 8), wherein decreased expression of PTEN polypeptide together with decreased phosphorylation of S6 ribosomal polypeptide in the sample, as compared to a control, identifies the glioma tumor as likely to respond or responsive to an mTOR inhibitor, and wherein decreased expression an of PTEN together with normal phosphorylation of S6 ribosomal polypeptide in the sample, as compared to a control, identifies the glioma tumor as not likely to respond or non-responsive to an mTOR inhibitor, and wherein normal or increased expression of PTEN and increased expression and/or activity of EGFR together with increased phosphorylation of AKT and/or phosphorylation of ERK identifies the glioma tumor as not likely to respond and/or non-responsive to an EGFR inhibitor.

Another preferred embodiment of the invention is a method for identifying a mammalian prostate tumor likely to respond or responsive to an EGFR polypeptide (SEQ ID NO: 7) inhibitor or an mTOR polypeptide (SEQ ID NO: 2) inhibitor, the method comprising examining a sample obtained from the tumor for: the expression of PTEN polypeptide (SEQ ID NO: 5); and the presence of at least one of, a phosphorylated S6 ribosomal polypeptide (SEQ ID NO: 1); a EGFR polypeptide (SEQ ID NO: 7); a phosphorylated AKT polypeptide (SEQ ID NO: 4); and a phosphorylated ERK polypeptide (SEQ ID NO: 8), wherein decreased expression of PTEN polypeptide together with decreased phosphorylation of S6 ribosomal polypeptide in the sample, as compared to a control, identifies the prostate tumor as likely to respond or responsive to an mTOR inhibitor, and wherein decreased expression an of PTEN together with normal phosphorylation of S6 ribosomal polypeptide in the sample, as compared to a control, identifies the prostate tumor as not likely to respond or non-responsive to an mTOR inhibitor, and wherein normal or increased expression of PTEN and increased expression and/or activity of EGFR together with increased phosphorylation of AKT and/or phosphorylation of ERK identifies the prostate tumor as not likely to respond and/or non-responsive to an EGFR inhibitor.

Another embodiment of the invention is a kit for characterizing a mammalian tumor or cell, the kit comprising: an antibody that binds PTEN (SEQ ID NO: 5); and/or an antibody that binds phosphorylated S6 ribosomal polypeptide (SEQ ID NO: 1); and/or an antibody that binds EFGR (SEQ ID NO: 7); and/or an antibody that binds phosphorylated AKT (SEQ ID NO: 4); and/or an antibody that binds phosphorylated AKT (SEQ ID NO: 8). Optionally the kit further includes an antibody that binds Ki-67 polypeptide (SEQ ID NO: 9), and/or p-H3 histone polypeptide (SEQ ID NO: 10) and/or caspase-3 polypeptide (SEQ ID NO: 11). Typically the kit further comprises a secondary antibody which binds to one of the primary antibodies directed to these polypeptides. Optionally the kit comprises a plurality of antibodies that bind to the various polypeptides.

### BRIEF DESCRIPTION OF THE DRAWINGS

FIG. 1 shows the immunohistochemical expression of PTEN, p-Akt, p-mTOR, p-FKHR and p-S6 in GBM tumor samples. (A) Representative images demonstrating PTEN protein loss in tumors cells with retention of staining in vascular endothelium (0), diminished PTEN staining relative to the endothelium (1), and no evidence of PTEN protein loss (2). NC is the negative control. (B) Staining for p-Akt, p-mTOR, p-FKHR and p-S6 scored on a scale of 2 (strong), 1 (mild) and 0 (negative). NC represents negative controls.

FIG. 2 shows the immunohistochemical expression of EGFR, EGFRvIII and p-Erk in GBM tumor samples. (A) Representative images demonstrating diffuse EGFR, EGFRvIII and p-Erk positivity (+). Representative images of tumors lacking EGFR, EGFRvIII and p-ERK expression are also shown (−). NC represents the negative controls.

FIGS. 3A and 3B provide an illustration of the interaction between members of the PI3K/Akt pathway and kinase inhibitors in GBM tumor samples. FIG. 3A shows that rapamycin inhibits S6 phosphorylation in glioblastoma in vivo. FIG. 3B shows that the rapamycin-mediated inhibition of S6 phosphorylation correlates with diminished tumor proliferation. In this Figure, Ki-67, a market of cellular proliferation was used to assess whether rapamycin-mediated inhibition of S6 had an effect on tumor growth.

FIG. 4 provides a schematic demonstrating the components of the PI3K signaling pathway.

FIGS. 5A-5E provide a validation of PTEN and the phospho-specific antibodies. A) PTEN expression in prostate cancer cell lines. Sections of cell pellets prepared from DU145 (PTEN Wild Type) and LNCaP Deletion of one allele and mutation in second allele). Note the lack of immunostaining in LNCaP cells. B) p-AKT, p-S6, p-FKHR and p-ERK immunostaining in a formalin fixed, paraffin embedded breast tumor section. The left panel shows positive staining with the antibody. There is no staining when a specific antigen peptide is used in combination with the respective phospho-specific antibodies, tight panel. C) Immunofluorescent detection of total AKT (red) or p-AKT Ser473 (green) in paraffin-embedded LNCaP cells, untreated (left) or treated with 30 μM LY294002 (right). Yellow represents overlay. Note the decreased intensity of phosphorylated AKT staining in the LY294002-treated cells, with unchanged expression of AKT. D) LNCaP cells were grown in culture and exposed either to vehicle or the PI3K inhibitor, LY294002 (30 μM), for 18 hours. Cell pellets were prepared, formalin fixed and embedded in paraffin, then cut for immunostaining with phospho-AKT, p-FKHR, p-S6 and p-ERK antibodies. The addition of LY2940002 resulted in decreased staining intensity of p-AKT, p-FKHR and p-S6K. The staining intensity of p-ERK before and after treatment remained unchanged. Next, p-ERK staining was examined in HT129 cells, by the addition of EGF (100 ng/ml for 20 minutes). This staining is subsequently reduced when a MEK inhibitor, UO-126 (20 μM for 20 minutes) is added to the induced cells, in contrast to lack of inhibition by LY294002. E). LNCaP cells were grown in culture and exposed to either vehicle or the mTOR inhibitor, CCI-779 (20 nM) for 18 hours and subsequently prepared as described in the material and methods section. Sections were stained with p-AKT and p-S6. The addition of CCI-779 resulted in decreased staining intensity of p-S6 but had no effect on p-AKT, which is consistent with mTOR being downstream of AKT but upstream of p-S6.

FIGS. 6A and 6B provide a definition of PTEN scoring criteria. A) Range of PTEN immunostaining. In our series, PTEN protein expression was found to be absent in 29% (Score=0, a), decreased compared to benign glands in 11% (Score=1, b) and equivalent to benign glands in 60% of tumors (Score=2, c). Arrows indicate staining characteristics. B) PI3K pathway activation in a patient with loss of PTEN expression. Serial sections from the same tumor show loss of PTEN expression results in the phosphorylation of p-AKT, p-S6 and p-FKHR. In this case, p-ERK is also active.

FIG. 7 provides a demonstration of the inter-relationships between signaling molecules in vivo. MDS analysis shows that PTEN protein and AKT phosphorylation are tightly linked, confirming what is known of their in vitro relationship.

### DETAILED DESCRIPTION OF THE INVENTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Ausubel et al., Current Protocols in Molecular Biology, Wiley Interscience Publishers, (1995). As appropriate, procedures involving the use of commercially available kits and reagents are generally carried out in accordance with manufacturer defined protocols and/or parameters unless otherwise noted.

“Mammal” for purposes of treatment or therapy refers to any animal classified as a mammal, including humans, domestic and farm animals, and zoo, sports, or pet animals, such as dogs, horses, cats, cows, etc. Preferably, the mammal is human.

The terms “cancer”, “cancerous”, or “malignant” refer to or describe the physiological condition in mammals that is typically characterized by unregulated cell growth. Examples of cancer include but are not limited to astrocytoma, blastoma, carcinoma, glioblastoma, leukemia, lymphoma and sarcoma. More particular examples of such cancers include breast cancer, ovarian cancer, colon cancer, colorectal cancer, rectal cancer, squamous cell cancer, small-cell lung cancer, non-small cell lung cancer, Hodgkin's and non-Hodgkin's lymphoma, testicular cancer, esophageal cancer, gastrointestinal cancer, renal cancer, pancreatic cancer, glioblastoma, cervical cancer, glioma, liver cancer, bladder cancer, hepatoma, endometrial carcinoma, salivary gland carcinoma, kidney cancer, liver cancer, prostate cancer, vulval cancer, thyroid cancer, hepatic carcinoma and various types of head and neck cancer.

“Growth inhibition” when used herein refers to the growth inhibition of a cell in vitro and/or in vivo. The inhibition of cell growth can be measured by a wide variety of methods known in the art. A “growth inhibitory agent” when used herein refers to a compound or composition which inhibits growth of a cell in vitro and/or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL®, and topo II inhibitors such as doxorubicin, epirubicin, daunorubicin, etoposide, and bleomycin. Those agents that attest G1 also spill over into S-phase attest, for example, DNA alkylating agents such as tamoxifen, prednisone, dacarbazine, mechlorethamine, cisplatin, methotrexate, 5-fluorouracil, and ara-C. Such agents further include inhibitors of cellular pathways associated with disregulated cell growth such as the PI3K/Akt pathway. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995).

“Treatment” or “therapy” refer to both therapeutic treatment and prophylactic or preventative measures. The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing tumor burden or volume, the time to disease progression (TTP) and/or determining the response rates (RR).

The term “antibody” is used in the broadest sense and specifically covets single monoclonal antibodies and antibody compositions with polyepitopic specificity (e.g. polyclonal antibodies) as well as antibody fragments so long as retain their ability to immunospecifically recognize a target polypeptide epitope.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being detected against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

As used herein, the term “polynucleotide” means a polymeric form of nucleotides of at least 10 bases or base pairs in length, either ribonucleotides or deoxynucleotides or a modified form of either type of nucleotide, and is meant to include single and double stranded forms of DNA and/or RNA. In the art, this term if often used interchangeably with “oligonucleotide”. A polynucleotide can comprise a nucleotide sequence disclosed herein wherein thymidine (T) can also be uracil (U); this definition pertains to the differences between the chemical structures of DNA and RNA, in particular the observation that one of the four major bases in RNA is uracil (U) instead of thymidine (T).

As used herein, the term “polypeptide” means a polymer of at least about 10 amino acids. Throughout the specification, standard three letter or single letter designations for amino acids are used. In the art, this term is often used interchangeably with “protein”.

As used herein, the term “inhibitor” encompasses molecules capable of inhibiting one or more of the biological activities of target molecules such as mTOR and/or EGFR polypeptide. Illustrative inhibitors include the targeted small-molecule inhibitors and antibody inhibitors disclosed herein as well as other inhibitors known in the art such as anti-sense polynucleotides and siRNA. Consequently one skilled in the art will appreciate that such inhibitors encompass molecules which inhibit both polynucleotide synthesis and/or function (e.g. antisense polynucleotide molecules) as well those which inhibit polypeptide synthesis and/or function (e.g. molecules which block phosphorylation and hence activity of a target polypeptide such as mTOR).

### Physiological Processes Pertinent to the Invention

Deregulated activation of the PI3K/Akt pathway is common in a variety of different cancers (see, e.g. Fresno Vara et al., Cancer Treat Rev. 30(2): 193-204 (2004; Mitsiades et al., Curr. Cancer Drug targets, 4(3): 235-256 (2004); Brader et al., Tumori, 90(1): 2-8 (2004); and Sansal et al., J. Clin. Oncol., 22(14): 2954-2963 (2004). An illustrative but non limiting list of cancers that exhibit deregulated activation of the PI3K/Akt pathway, include glioblastomas and cancers of the prostate (see, e.g., Vivanco et al., Nat Rev Cancer. 2: 489-501, 2002; Feldkamp et al., Journal of Neurooncology 35: 223-248, 1997; Mischel et al., Brain Pathology, January; 13(1):52-61 2003) as well as cancers of the bile duct (see, e.g. Tanno et al., Cancer Res., 64(10): 3486-3490 (2004)), bladder (see, e.g. Riegler-Christ et al., Oncogene, 23(27): 4745-53 (2004), breast (see, e.g. DeGraffenried et al., Ann. Oncol., 15(10): 1510-1516 (2004)), colon (see, e.g. Itoh et al., Cancer, 94(12): 3127-34 (2004)), endometrium (see, e.g. Gagnon et al., Int. J. Oncol., 23(2): 803-10 (2003), leukocytes (see, e.g. Cuni et al., Leukemia, 18(8): 1438-40 (2004); Kubota et al., Leukemia, 18(8): 1391-400 (2004); and Tabelli et al., Br. J. Haematol. 126(4): 574-82 (2004)), liver (see, e.g. Wang et al., Genes Devel. 18(8): 912-25 (2004)), lung (see, e.g. Sithanandam et al., Carcinogenesis 24(10): 1581-92 (2003); and Cappuzzo et al., J. Natl. Cancer Inst. 96(15): 1133-1141 (2004)), melanocytes (see, e.g. Dhawan et al., 62(24): 7335-42 (2002)) ovary (see, e.g. Altomare et al., Oncogene 23(24): 5853-7 (2004)) pancreas (see, e.g. Perugini et al., J. Surg. Res. 90(1): 39-44 (2000) and thyroid (see, e.g. Vasko et al., J. Med. Genet. 41(3): 161-70 (2004)). Consequently, the assessment of this pathway is critical for stratifying patients for targeted kinase inhibitor therapy. The disclosure provided herein identifies a series of biomarkers that are associated with deregulated activation of the PI3K/Akt pathway as well as optimized methods for examining these markers. Consequently, the disclosure provided herein allows the examination of this pathway in cancer. Significantly, the disclosed methods for examining these markers are useful with a wide variety of tissue samples including formalin fixed, paraffin embedded biopsy samples. Various aspects of this disclosure are described in Choe et al., Cancer Res. 2003 Jun. 1; 63(11):2742-6.

As noted above, the disclosure provided herein identifies a series of biomarkers that are associated with deregulated activation of the PI3K/Akt pathway, a pathway whose deregulated activation is common in cancers such as gliomas. The disclosure provided herein further provides optimized methods for examining these biomarkers. Consequently, the disclosure allows the examination of the activation status of these biomarkers in a variety of cancers. As the art that teaches that this growth related pathway is common pathway that is disregulated in a wide variety of human cancers, artisans understand that the methods and materials disclosed herein can be universally applied to examine this pathway in all cancers in which the deregulated activation of the PI3K/Akt pathway is observed.

Significantly, the disclosed methods for examining these biomarkers are useful with a wide variety of tissue samples including formalin fixed, paraffin embedded biopsy samples. As disclosed herein, these markers can be examined using a panel of antibodies such as phospho-specific antibodies. In these methods, a mammalian cell such as a cell derived from a formalin fixed, paraffin embedded glioblastoma multiforme biopsy sample can be examined for evidence of Akt pathway activation by examining a tissue sample containing this cell for the presence of the various target molecules disclosed herein including phosphorylated polypeptides. Certain embodiments of the invention identify and/or assess a therapeutic agent that may be used to treat the glioblastoma such as rapamycin or an analogue thereof or an EGFR inhibitor such as ZD-1839 or an analogue thereof.

As noted above, the invention disclosed herein provides methods and immunohistochemical reagents that can be used to identify the activation state of the PI3K/Akt signaling pathway in clinical samples such as prostate and glioblastoma biopsy samples. These methods and reagents identify a coordinate regulation of the Akt/mTOR signaling pathway in response to loss of the PTEN tumor suppressor gene. As specific kinase inhibitors that target this pathway are currently in development (see, e.g., Neshat et al., Proc Natl Acad Sci USA. 98: 10314-9, 2001), and further because this mutation is common in glioblastoma and prostate cancer, this disclosure provides an important clinical tool for selecting patients for appropriate therapy. In this context, the invention can be practiced by performing immunohistochemical analysis on routinely processed patient biopsy samples. The results of these assays can be used as criteria for inclusion in clinical trials, and to assess outcome differences in patients in which this pathway is deregulated.

The methods and reagents disclosed herein can be used to determine the activation state of biomarker polypeptides such as Akt and its downstream effectors such as mTOR, ERK, Forkhead and S6-kinase on routinely processed patient biopsy samples (e.g. glioblastoma samples) and this information can be used to select patients for therapy with targeted pathway inhibitors. As disclosed herein, the invention has been tested on biopsies from patients suffering from glioblastoma as well as patients suffering from cancers of the prostate. The results demonstrate clear coordinate regulation of Akt, mTOR, forkhead and S6-kinase, and their association with PTEN loss. A detailed discussion of the biomarkers and the physiological processes pertinent to the invention is provided below.

Activation of PI3K by growth factor signaling catalyzes the formation of phosphatidylinositol triphosphate (PIP3) by addition of a phosphate group to phosphoinositol bisphosphate (PIP2). PIP3 catalyzes the activation of the Akt kinase (and its downstream effectors mTOR, forkhead and S6-kinase), which promote cell proliferation and survival. The PTEN tumor suppressor gene encodes a phosphatase that removes the phosphate group from PIP3, thereby regulating the activation state of this pathway. PTEN loss results in constitutive signaling through PIP3, and hence unregulated activation of the Akt pathway.

PTEN is lost in many types of cancer including glioblastomas and cancers of the prostate. In addition, the Akt pathway is dysregulated in many other cancers. PTEN-deficient cancer cells are dramatically more sensitive to inhibition of the Akt pathway at the level of mTOR (see, e.g., Neshat et al., Proc Natl Acad Sci USA. 98: 10314-9, 2001), than PTEN wild-type cells, including non-cancerous cells. Therefore, mTOR inhibitors can be a highly selective and effective therapy for patients whose tumors have PTEN loss and Akt pathway activation. All prior knowledge of the PTEN/PI3K/Akt pathway is based on biochemical data and genomic analysis, which are not feasible as a clinical screening tool. Currently, there are no methods for detection of the activation state of this pathway in routinely processed formalin-fixed, paraffin-embedded patient biopsy samples. Consequently, the ability to identify the activation state of this pathway in such clinical samples, and to select patients for its inhibition is a valuable diagnostic tool. This is also valuable tool for the analyses of inhibitors that target this pathway.

As specifically disclosed herein we demonstrate that PI3′K/Akt pathway activation can be detected in routinely processed patient biopsies. We demonstrate that PTEN loss is significantly correlated with Akt activation, which is significantly associated with activation of downstream effectors mTOR, S6 and FKHR. We have also shown that PTEN loss is not the only mechanism of PI3′K/Akt pathway activation, and demonstrated that EGFR and EGFRvIII co-expression are significantly associated with activation of this pathway. Finally, we demonstrate that PI3K/Akt and Erk pathway activation have significant impact on cancer progression and survival. These data provides evidence that this set of tools can be used to stratify cancer patients for targeted molecular therapy.

The epidermal growth receptor factor receptor contributes to the malignant phenotype of human glioblastomas (see, e.g. Thomas et al., Int J. Cancer. 2003 Mar. 10; 104(1):19-27). Studies in SKMG-3 cells, a GBM cell line that maintains EGFR gene amplification in vitro demonstrate that EGF treatment stimulated phosphorylation of the EGFR as well as the downstream effectors Erk, AKT1, stat3 and c-Cbl. Under minimal growth conditions, unstimulated SKMG-3 cells contain constitutively phosphorylated Erk and AKT-1. The EGFR kinase inhibitor PD158780 reduces the constitutive phosphorylation of the receptor and Erk but not that of AKT1. In contrast, inhibition of phosphatidylinositol-3-kinase (PI3K) blocks the constitutive phosphorylation of Erk and AKT-1 but not the EGFR. The results provide evidence that signals from overexpressed EGFR contribute to the constitutive phosphorylation of Erk, but these signals may not requited for the constitutive activation of PI3K or AKT1. See, e.g. Thomas et al., Int J Cancer. 2003 Mar. 10; 104(1):19-27.

In addition, EGFR appears to play an important role in the pathogenesis of colorectal cancer as shown for example by studies of the EGFR tyrosine kinase inhibitor ZD1839 in metastatic colorectal cancer patients in which serial biopsies were taken pre- and posttreatment to assess biological activity (see, e.g. Daneshmand Clin Cancer Res. 2003 July; 9(7):2457-64). In these studies, paired biopsies were obtained from colorectal cancer patients before and after treatment. Posttreatment samples showed a statistically significant reduction in cancer cell proliferation. While all pretreatment samples showed strong staining for EGFR, loss of immunohistochemical staining for activated EGFR, phosphorylated Akt, and phosphorylated ERK in cancer cells was observed in some patients after treatment. See e.g., Daneshmand Clin Cancer Res. 2003 July; 9(7):2457-64.

The PI3′K/Akt pathway is commonly deregulated in GBMs, but its identification in routine biopsies has presented a challenge. In the face of new kinase inhibitors that target this pathway, the need for an assay that can be used to stratify patients for therapy has become critical. As disclosed herein, we demonstrate that activation of the PI3′K/Akt pathway can be detected by immunohistochemistry using a panel of phospho-specific antibodies. We show that 38% of untreated primary GBMs demonstrate evidence of PTEN protein loss, and that this is significantly associated with Akt activation. We further demonstrate that phosphorylation of Akt is significantly correlated with phosphorylation of downstream effectors mTOR, FKHR and S6. We show that PTEN loss is not the only mechanism underlying Akt pathway activation; phosphorylation of Akt, mTOR, S6 and FKHR are also associated with co-expression of EGFR and its constitutively active variant EGFRvIII. Finally, we demonstrate that activation of the PI3′K/Akt and Erk pathways is associated with shorter time to progression and diminished overall survival in GBM patients.

The disclosure provided herein demonstrates that PI3′K/Akt pathway activation can be detected in paraffin-embedded biopsy samples, and provides evidence that PTEN loss is highly correlated with Akt pathway activation in primary GBMs. These results also provide evidence that co-expression of EGFR and EGFRvIII can activate the PI3′K pathway in GBMs with normal PTEN immunohistochemical expression. The results further provide evidence that activation of these signaling pathways has considerable impact on GBM patient progression and survival.

The disclosure provided herein specifically demonstrates that the activation of the PI3′K/Akt pathway can be detected with phospho-specific antibodies in routinely processed patient biopsies. In one illustrative embodiment of the invention, the disclosed methods and materials are used to examine glioblastomas. In another illustrative embodiment of the invention that is disclosed herein, the disclosed methods and materials are used to examine cancers of the prostate. In the context of glioblastomas, we show that PTEN-deficient GBMs have coordinated activation of the Akt pathway and its downstream effectors mTOR, FKHR and S6. We also show that GBMs co-expressing EGFR and EGFRvIII have activation of the PI3′K/Akt and Erk signaling pathways. Finally, we demonstrate that activation of these signal transduction pathways has prognostic importance. For example, primary GBM patients whose tumors are activated downstream of Akt, or at the level of ERK, have significantly shorter time to tumor progression and significantly diminished overall survival. These results define molecular subtypes of GBMs and may be used to stratify patients for targeted molecular therapy.

As disclosed in detail below, in illustrative analytical methods we generated a tissue microarray from 45 untreated primary GBM patient biopsies and analyzed the immunohistochemical expression of p-Akt and downstream effectors p-mTOR, p-FKHR and p-S6, as well as p-Erk. EGFR, EGFRvIII expression, and PTEN loss, all of which can promote activation of the PI3′K/Akt pathway, were also analyzed and association with PI3′K/Akt and Erk pathway activation were determined. The prognostic implications of PI3′K/Akt and Erk pathway activation were also analyzed.

In out analysis the loss of PTEN immunohistochemical expression was detected in 38% of GBMs. Diminished PTEN protein expression was significantly associated with phosphorylation of Akt (p<0.00001) and downstream effectors mTOR (p=0.04), FKHR (p=0.006) and S6 (p=0.001). PTEN protein loss was not associated with Erk activation, which is independent of PI3′K/Akt signaling. PTEN protein loss was not the only route to PI3′K/Akt pathway activation; co-expression of EGFR and EGFRvIII was significantly correlated with expression of p-Akt (p=0.06), p-mTOR (p=0.001), p-FKHR (p=0.002) and p-S6 (p=0.001) in GBMs with normal PTEN protein expression. EGFR and EGFRvIII co-expression was also associated with Erk activation (p=0.007). Concurrent phosphorylation of mTOR, FKHR and S6, was significantly associated with shorter time to progression (p=0.002) and decreased overall survival (p=0.02), as was Erk activation (p=0.04).

As noted above, the methods disclosed herein typically employ immunohistochemical analysis. Immunohistochemical analysis requires a subjective determination by pathologists. Proteomic approaches have the potential to be a more objective and sensitive methods and may become clinically feasible in the future (see, e.g., Liotta et al., Jama. 286: 2211-4, 2001; Liotta et al., Breast Cancer Res. 2: 13-4, 2000; Petricoin et al., Lancet. 359: 572-7, 2002; Petricoin et al., Nat Rev Drug Discov. 1: 683-95, 2002). However, the current need to stratify patients for targeted therapy, and to assess molecular correlates of response to experimental targeted agents, dictates that we develop assays that work on routinely processed biopsy samples using currently accessible methods. Activated Akt can be detected by immunohistochemistry done on patient biopsies, and it has been suggested that it may have biological or prognostic implications (see, e.g., Gupta et al., Clin Cancer Res. 8: 885-92, 2002; Malik et al., Clin Cancer Res. 8: 1168-71, 2002). Complementary to previous studies, we demonstrate here that a panel of phospho-specific antibodies can be used to detect p-Akt and its downstream effectors in order to map PI3′K/Akt pathway activation. The high level of association between the downstream effector activation and Akt phosphorylation, provides evidence that we have accurately assessed this pathway. Further, our data showing that PI3′K/Akt pathway activation is associated with PTEN protein loss (see, e.g., Neshat et al., Proc Natl Acad Sci USA. 98: 10314-9, 2001; Ermoian et al., Clin Cancer Res. 8: 1100-6, 2002) or EGFR/EGFRvIII signaling, are highly consistent with recent in vitro and in animal models (see, e.g., Davies et al., Cancer Res. 59: 2551-6, 1999; Davies et al., Cancer Res. 58: 5285-90, 1998; Lorimer et al., Biochim Biophys Acta. 1538: 1-9, 2001; Moscatello et al., J Biol Chem. 273: 200-6, 1998), including a recent biochemical demonstration that PTEN protein level is inversely correlated with Akt activation in GBM patient biopsies (see, e.g., Ermoian et al., Clin Cancer Res. 8: 1100-6, 2002).

Our finding that ERK and PI3′K/Akt pathway activation were associated with shorter time to progression and decreased overall survival is the first demonstration that pathway activation may have an impact on GBM patient prognosis. The data presented herein provides evidence that pathway activation status conveys important prognostic information. It is surprising that Akt activation was not significantly associated with progression or survival, while downstream activation at the level of mTOR, S6 and FKHR was. This result raises two possibilities. Either the p-Akt antibody is a less sensitive tool for detecting PI3′K/Akt pathway activation than is the panel of downstream phospho-specific antibodies. Alternatively, convergent inputs to mTOR, FKHR and S6 downstream of Akt, or in and Akt-independent fashion, may play an important role in modulating the biological behavior of GBMs. In line with this, concurrent Erk and Akt-mediated signaling may be required for optimal activation of p70 S6 kinase, and formation of p-S6 (see, e.g., Iijima et al., J Biol. Chem. 277: 23065-75, 2002; Shi et al., J Biol Chem. 277: 15712-20, 2002). Similarly, Akt-independent mechanisms of mTOR and FKHR phosphorylation have been demonstrated (see, e.g., Gingras et al., Genes and Development. 15: 807-826, 2001; Burgering et al., Trends Biochem Sci. 27: 352-60, 2002). Using the disclosure provided herein and methods typically employed in the art one can determine whether these additional inputs play a role in modulating GBM behavior. For additional discussions of EGFR and Akt activity and inhibitors thereof, see, e.g. Bianco et al., Oncogene, 2003 May 8; 22(18):2812-22; Yakes et al., Cancer Res. 2002 Jul. 15; 62(14):4132-41; and She et al., Clin Cancer Res. 2003 Oct. 1; 9(12):4340-6, the contents of which are incorporated herein by reference

While the sample size of 45 patients is relatively modest, it was large enough to provide robust associations between PTEN loss and PI3′K/Akt pathway activation. Only untreated primary GBM patients were included in this study. Since treatment itself may modulate Erk and PI3′K/Akt pathway activation, this study design enabled us to better assess the association between pathway activation and upstream molecular events. Using the disclosure provided herein and methods typically employed in the art one can perform both retrospective, and prospective analyses of GBM patients (both treated and untreated) to further quantify the prognostic implications of pathway activation and to identify molecular correlates of response to therapy.

In order to address any subjectivity of immunohistochemical analysis all immunostains were interpreted independently by two neuropathologists, and by one of the neuropathologists at independent occasions, and the inter-rater and intra-rater agreement were high for all stains. This provides evidence that interpretation of these phospho-specific antibodies will be reproducible between independent pathologists. In the future, more objective methods such as proteomic analysis can replace these tools (see, e.g., Liotta et al., Jama. 286: 2211-4, 2001; Petricoin et al., Nat Rev Drug Discov. 1: 683-95, 2002). Nonetheless, the data presented here provides evidence that we can accurately assess these pathways using currently available methods, and provides evidence that one can stratify patients for therapy.

GBMs are among the most heterogeneous tumors, as has been previously shown (see, e.g., Cheng et al., J Neuropathol Exp Neurol. 58: 120-8, 1999; Jung et al., J Neuropathol Exp Neurol. 58: 993-9, 1999). This poses a problem for assessment of molecular alterations in GBMs, as well as for stratification of patients for targeted inhibitor therapy. Using the disclosure provided herein and methods typically employed in the art one can directly determine the extent of intra-tumor molecular heterogeneity for PTEN, EGFR and EGFRvIII and assess the impact of this on pathway activation, prognosis and response to therapy.

As noted above, typical embodiments of the invention examine cellular pathways in the family of tumors termed “gliomas”. Briefly, the brain contains two major cell types: neurons and glia. Glial cells give rise to the family of tumors termed “gliomas”. There are several distinct types of tumors within this glioma grouping. These can range from very benign, slow-growing tumors to rapidly enlarging, highly malignant cancerous types. The most commonly occurring tumors within the glioma family are astocytomas, oligodendroglioma and ependymomas. In addition, some patients may have tumors with a mixed appearance. Astrocytomas are the most common type of glioma. These are tumors that occur within the brain tissue itself. Like all gliomas, astrocytomas can be located either superficially or deep within the brain and can affect critical structures. As they arise from the astrocyte cells (which serve as supporting elements of the brain), astrocytomas are generally infiltrative in nature.

As discussed in detail below, the World Health Organization (WHO) grading scheme is used to characterize this group of tumors. Briefly, in the World Health Organization grading system, grade I tumors are the least malignant. These tumors grow slowly and microscopically appear almost normal; surgery alone may be effective. Grade I tumors are often associated with long-term survival. Grade II tumors grow slightly faster than grade I tumors and have a slightly abnormal microscopic appearance. These tumors may invade surrounding normal tissue, and may recur as a grade II or higher tumor. Grade III tumors are malignant. These tumors contain actively reproducing abnormal cells and invade surrounding normal tissue. Grade III tumors frequently recur, often as grade IV tumors. Grade IV tumors are the most malignant and invade wide areas of surrounding normal tissue. These tumors reproduce rapidly, appear very unusual microscopically and are necrotic (have dead cells) in the center. Grade IV tumors cause new blood vessels to form, to help maintain their rapid growth. Glioblastoma multiforme is the most common grade IV tumor. For additional information see, e.g. Tatter S B, Wilson C B, Harsh G R IV. Neuroepithelial tumors of the adult brain. In Youmans J R, ed. Neurological Surgery, Fourth Edition, Vol. 4: Tumors. W.B. Saunders Co., Philadelphia, pp. 2612-2684, 1995; Kleihues P, Burger P C, Scheithauer B W. The new WHO classification of brain tumours. Brain Pathology 3:255-68, 1993; Lopes M B S, VandenBerg S R, Scheithauer B W; The World Health Organization classification of nervous system tumors in experimental neuro-oncology. In A. J. Levine and H. H. Schmidek, eds. Molecular Genetics of Nervous System Tumors Wiley-Liss, New York, pp. 1-36, 1993.

Low-grade astrocytomas (Grades I/IV or II/IV) are termed benign and occur generally in children or young adults. These tumors carry a better prognosis than higher grade astrocytomas. Although the management of these low-grade astrocytomas can be controversial, those tumors which are surgically accessible are usually resected. One of the concerns with low-grade astrocytomas in adults is that they can undergo a malignant transformation and change into a higher-grade, or malignant tumor. The methods of the invention can be used to monitor such transformations. In astrocytomas grade I, normal karyotype is observed most frequently; among the cases with abnormal karyotypes, the most frequent chromosomal abnormalities loss of the X and Y sex-chromosomes; loss of 22q is found in 20-30% of astrocytomas; other abnormalities observed in low grade tumors include gains on chromosome 8q, 10p, and 12p, and losses on chromosomes 1p, 4q, 9p, 11p, 16p, 18 and 19.

Anaplastic astrocytomas (Grade III/IV) are more aggressive tumors and, as such, are usually treated in a more radical fashion. In anaplastic astrocytomas, chromosome gains or losses are frequent: trisomy 7 (the most frequent), loss of chromosome 10, loss of chromosome 22, loss of 9p, 13q; other abnormalities, less frequently described are: gains of chromosomes 1q, 11q, 19, 20, and Xq. Glioblastoma multiforme (Grade IV/IV) is the most malignant form of astrocytomas. Although these tumors can occur at almost any age, the peak incidence is between 50 and 70 years old. Glioblastoma multiforme (GBM) is also called a high-grade glioma and is graded by pathologists as Grade IV/IV astrocytoma. These tumors mostly occur in adults with the peak incidence between 50 and 70 years of age. Generally the time from the onset of symptoms to diagnosis is relatively short, usually just a few weeks. Glioblastomas typically show several chromosomal changes: by frequency order, gain of chromosome 7 (50-80% of glioblastomas), double minute chromosomes, total or partial monosomy for chromosome 10 (70% of tumors) associated with the later step in the progression of glioblastomas partial deletion of 9p is frequent (64% of tumors): 9pter-23; partial loss of 22q in 22q13 is frequently reported loss or deletion of chromosome 13, 13q14-q31 is found in some glioblastomas trisomy 19 was reported in glioblastomas by cytogenetic and comparative genomic hybridization (CGH) analysis; the loss of 19q in 19q13.2-qter was detected by loss of heterozygosity (LOH) studies in glioblastomas deletion of chromosome 4q, complete or partial gains of chromosome 20 has been described; gain or amplification of 12q14-q21 has been reported the loss of chromosome Y might be considered, when it occurs in addition to other clonal abnormalities.

Oligodendrogliomas are benign, slow growing tumors that occur usually in young adults. Often these are located within the frontal lobes which can allow for a safe, complete operative resection. Many oligodendrogliomas contain calcium (little specks of bone) seen best on CT scans.

In addition to glioblastomas, the methods of the invention are applicable to a wide variety of other cancers where disregulation of the PI3K/Akt pathway is associated with a concurrent disregulation in cellular growth. For example, in yet another illustrative embodiment of the invention, the disclosed methods and materials are used to examine cancers of the prostate. Utilizing prostate cancer tissue biopsies and/or microarrays, we show that this pathway can be interrogated in vivo, using immunohistochemical assays and identify a subset of patients who exhibit complete loss of PTEN protein expression. Loss of PTEN protein expression was significantly correlated with activation of AKT. Activation of AKT was in turn, associated with phosphorylation of S6, in keeping with what is known of the relationships between these signaling molecules. In terms of PTEN biology, it has been shown in genetic systems (i.e. conditional knock-outs) that the dosage of PTEN loss correlates with murine prostate cancer progression. PTEN loss, when examined by Pearson and Multivariate analysis, is significantly associated with phosphorylation of AKT. The phospho-specific antibodies disclosed herein were also used to follow changes in phosphorylation status of these proteins in a prostate cancer cell line, before and after treatment with CCI-779. This highlights the utility of these antibodies to confirm target inhibition in patient samples post treatment.

As noted above, typical embodiments of the invention examine cellular pathways in the family of tumors termed “prostate cancers”. In this context, cancers of the prostate are typically categorized into stages. In stage T1 (Stage A), the tumor is located within the prostate only. It causes no symptoms and is too small to be felt during a digital rectal exam (DRE) or to be seen on an imaging scan. These tumors are usually found by chance during surgery for some other prostate disease, or by following up on screening tests that measure PSA. In stage T2 (Stage B), the tumor is still located within the prostate but is large enough to be felt during a DRE. There are often no symptoms. In Stage T3 (Stage C), the tumor has spread from the prostate into the immediately surrounding tissue, possibly including the seminal vesicles. In stage T4, the tumor is still within the pelvic region but may have spread farther to areas of the bladder or rectum. In stage N+ (Stage D1), prostate cancer is described as N+ if prostate cancer cells are detected in the lymph nodes in the pelvic area. In stage M+ (Stage D2), a tumor that is M+ has spread beyond the pelvic area to other parts of the body. Bone pain, weight loss, and tiredness are common symptoms.

Cancers of the prostate are also categorized into “grades” which describe the way the cancer tissue looks when seen under a microscope. The most common system used in the USA to grade the appearance of prostate cancer tissue is called the Gleason grading system. This grading system is based on a number range from 2 to 10. The lower the number, the lower the grade, and the slower the cancer is growing. The higher the score, the higher the grade of the tumor. High-grade tumors grow more quickly than low-grade tumors, and are more likely to spread to other parts of the body. Grades under 4 mean that the cancer cells look similar to your normal cells, and the cancer is likely to be less aggressive. Grades 5 to 7 are in the intermediate range. This means that the cancer cells do not look like normal cells, and are more likely to be aggressive and grow faster. Grades 8 to 10 indicate that the cancer cells are more likely to be very aggressive in growth.

Certain embodiments of the invention include methods to obtain information used to identify a therapeutic agent for treating a cancer such as a glioblastoma or a prostate cancer in a human. For example, methods of the invention examine the levels of certain polypeptides (e.g. PTEN) and/or the phosphorylation state of certain polypeptides (e.g. S6) to obtain information on how the cancer cell will respond to rapamycin or a rapamycin analog. Rapamycin (also known as sirolimus or rapammune) is a macrolide, related to cyclosporin with immunosuppressive properties and antiproliferative activity in various human tumor cells lines and tumor xenograft models. Both rapamycin and rapamycin analogues with more favorable pharmaceutical properties, such as SDZ-RAD, CCI-779, RAD 001, Everolimus (Certican) and AP23573, are highly specific inhibitors of mTOR. As noted herein the mammalian target of rapamycin (mTOR) is a downstream effector of the phosphatidylinositol 3-kinase (PI3K)/Akt (protein kinase B) signaling pathway that mediates cell survival and proliferation, and consequently is a target for anticancer therapeutic development. In essence, rapamycin and rapamycin analogues gain function by binding to the immunophilin FK506 binding protein 12 and the resultant complex inhibits the activity of mTOR. Because mTOR activates both the 40S ribosomal protein S6 kinase (p70s6k) and the eukaryotic initiation factor 4E-binding protein-1, rapamycin-like compounds block the actions of these downstream signaling elements, which results in cell cycle arrest in the G1 phase. Rapamycin and its analogues also prevent cyclin-dependent kinase (CDK) activation, inhibit retinoblastoma protein phosphorylation, and accelerate the turnover of cyclin D1, leading to a deficiency of active CDK4/cyclin D1 complexes, all of which potentially contribute to the prominent inhibitory effects of rapamycin at the G1/S boundary of the cell cycle. Rapamycin and rapamycin analogues have demonstrated impressive growth-inhibitory effects against a broad range of human cancers. For example, as noted herein, mammalian target of rapamycin (mTOR) modulates key signaling pathways that promote uncontrolled proliferation of glioblastoma multiforme. In this context the methods of the invention can be used to examine the PI3K/Akt pathway and then select an appropriate therapeutic agent in cells having a deregulated PI3K/Akt pathway (e.g. rapamycin). For discussions of Rapamycin and its analogs, see, e.g. Mita et al., Clin Breast Cancer 2003 June; 4(2):126-37; Hosoi et al., Mol Pharmacol. 1998 November; 54(5):815-24; Hidalgo et al., Oncogene. 2000 Dec. 27; 19(56):6680-6; Alexandre et al., Bull Cancer. 1999 October; 86(10):808-11; and Eshleman et al., Cancer Res. 2002 Dec. 15; 62(24):7291-7.

Overexpression of epidermal growth factor receptor (EGFR) is also observed in a wide variety of cancers such as glioma and has frequently been correlated with poor prognosis, thus stimulating efforts to develop new cancer therapies that target EGFR. Monoclonal antibodies and tyrosine kinase inhibitors specifically targeting EGFR are the most well-studied and hold substantial promise of success. Several compounds of monoclonal antibodies and tyrosine kinase inhibitors targeting EGFR have been studied and clinical trials are now underway to test the safety and efficacy of these targeting strategies in a variety of human cancers. Compounds that target the extracellular ligand-binding region of EGFR include antibodies such as Cetuximab (also known as Erbitux or IMC-C225). Other compounds such as tyrosine kinase inhibitors which target the intracellular domain of EGFR, include ZD-1839 (also known as gefitinib or Iressa), OSI-774 (also known as Erlotinibor or Tarceva), PD-153053, PD-168393 and CI-1033, have been studied in clinical settings alone or in combination with radiation or chemotherapy. In addition, compounds such as h-R3, ABX-EGF, EMD-55900 and ICR-62 have proved to be effective in targeting malignant cells alone or in combination with traditional therapies. The effects of ZD 1839 (Iressa) is currently being studied in clinical trails for patients with glioblastoma multiforme. In this context the methods of the invention can be used to examine the PI3K/Akt pathway and then select an appropriate therapeutic agent in cells that do not have a deregulated PI3K/Akt pathway (e.g. an EGFR inhibitor). For discussions of EGFR inhibitors see, e.g. Khalil et al., Expert Rev Anticancer Ther. 2003 June; 3(3):367-80; Chakravarti et al., Int J Radiat Oncol Biol Phys. 2003 Oct. 1; 57(2 Suppl):S329; Wissner et al., Bioorg Med Chem Lett. 2002 Oct. 21; 12(20):2893-7; Ciardiello et al., Expert Opin Investig Drugs, 2002 June; 11(6):755-68; De Bono et al., Trends Mol Med. 2002; 8(4 Suppl):S19-26; and Cohen, Clin Colorectal Cancer. 2003 February; 2(4):246-51.

### Typical Methods of the Invention

The invention disclosed herein has a number of embodiments. Illustrative embodiments of the invention include methods which examine tumor samples such as formalin fixed, paraffin embedded glioblastoma multiforme or prostate cancer biopsy samples for evidence of deregulated activation of the PI3K/Akt pathway. These methods involve examining the presence and/or phosphorylation status of the disclosed biomarkers that are associated with this pathway in order to identify and/or assess a therapeutic agent that may be useful in the treatment of such a cancer. As disclosed herein, the presence and/or phosphorylation status of the disclosed biomarkers serves as a market or proxy of pathway activity.

Typically, the methods of the invention are used in evaluating the whether a tumor such as a prostate cancer or glioma is likely to respond (i.e. is likely to exhibit growth inhibition) when contacted with an mTOR inhibitor or an EGFR inhibitor. In such embodiments, the presence and/or phosphorylation status of a biomarker polypeptide that is associated with the activation of a pathway (e.g. a phosphorylated S6 ribosomal polypeptide (SEQ ID NO: 1)) is examined to determine if the pathway is disregulated in that tumor and is therefore susceptible to inhibition by a inhibitor known to target that pathway. In such embodiments, the tumor is examined prior to its exposure to the inhibitor. Alternatively, the methods evaluate whether a tumor such as a glioma or prostate cancer is responsive (i.e. exhibits growth inhibition) to an mTOR inhibitor or an EGFR inhibitor. In such embodiments, the activity of a biomarker polypeptide that is associated with the activation of a pathway (e.g. a phosphorylated S6 ribosomal polypeptide (SEQ ID NO: 1)) is examined after the tumor is exposed to the inhibitor to determine if the biomarkers in the pathway respond to exposure to the inhibitor. As the art that teaches that this growth related pathway is common pathway that is disregulated in a wide variety of human cancers. Consequently, artisans understand that the methods and materials disclosed herein can be universally applied to examine this pathway in all cancers in which the deregulated activation of the PI3K/Akt pathway is observed. In this context, while the use of the disclosed methods and materials in the examination of gliomas and cancers of the prostate represent preferred embodiments of the invention, artisans understand that these are illustrative embodiments and that these methods and materials can be applied to a wide variety of human cancers.

One such embodiment of the invention is a method for identifying a mammalian glioma (e.g. glioblastoma multiforme) tumor likely to respond, is responsive to an EGFR polypeptide (SEQ ID NO: 7) inhibitor or an mTOR polypeptide (SEQ ID NO: 2) inhibitor, the method comprising examining a sample obtained from the tumor for: the expression of PTEN polypeptide (SEQ ID NO: 5); and the presence of at least one of, a phosphorylated S6 ribosomal polypeptide (SEQ ID NO: 1); a EGFR polypeptide (SEQ ID NO: 7); a phosphorylated AKT polypeptide (SEQ ID NO: 4); and a phosphorylated ERK polypeptide (SEQ ID NO: 8), wherein decreased expression of PTEN polypeptide together with decreased phosphorylation of S6 ribosomal polypeptide in the sample, as compared to a control, identifies the glioma tumor as likely to respond or responsive to an mTOR inhibitor, and wherein decreased expression an of PTEN together with normal phosphorylation of S6 ribosomal polypeptide in the sample, as compared to a control, identifies the glioma tumor as not likely to respond or non-responsive to an mTOR inhibitor, and wherein normal or increased expression of PTEN and increased expression and/or activity of EGFR together with increased phosphorylation of AKT and/or phosphorylation of ERK identifies the glioma tumor as not likely to respond and/or is nonresponsive to an EGFR inhibitor. Optionally, the phosphorylation of S6 ribosomal polypeptide is determined subsequent to contacting the tumor or sample with an mTOR inhibitor and/or the phosphorylation of AKT and/or ERK is determined subsequent to contacting the tumor or sample with an EGFR inhibitor. In illustrative embodiments, the mTOR inhibitor is rapamycin, SDZ-RAD, CCI-779, RAD 001, or AP23573 and the EGFR inhibitor is ZD-1839, OSI-774, PD-153053, PD-168393, IMC-C225 or CI-1033.

Another embodiment of the invention is a method for identifying a mammalian prostate tumor likely to respond, is responsive to an EGFR polypeptide (SEQ ID NO: 7) inhibitor or an mTOR polypeptide (SEQ ID NO: 2) inhibitor, the method comprising examining a sample obtained from the tumor for: the expression of PTEN polypeptide (SEQ ID NO: 5); and the presence of at least one of, a phosphorylated S6 ribosomal polypeptide (SEQ ID NO: 1); a EGFR polypeptide (SEQ ID NO: 7); a phosphorylated AKT polypeptide (SEQ ID NO: 4); and a phosphorylated ERK polypeptide (SEQ ID NO: 8), wherein decreased expression of PTEN polypeptide together with decreased phosphorylation of S6 ribosomal polypeptide in the sample, as compared to a control, identifies the prostate tumor as likely to respond or responsive to an mTOR inhibitor, and wherein decreased expression an of PTEN together with normal phosphorylation of S6 ribosomal polypeptide in the sample, as compared to a control, identifies the prostate tumor as not likely to respond or non-responsive to an mTOR inhibitor, and wherein normal or increased expression of PTEN and increased expression and/or activity of EGFR together with increased phosphorylation of AKT and/or phosphorylation of ERK identifies the prostate tumor as not likely to respond and/or is non-responsive to an EGFR inhibitor. Optionally, the phosphorylation of S6 ribosomal polypeptide is determined subsequent to contacting the tumor or sample with an mTOR inhibitor and/or the phosphorylation of AKT and/or ERK is determined subsequent to contacting the tumor or sample with an EGFR inhibitor. In illustrative embodiments, the mTOR inhibitor is rapamycin, SDZ-RAD, CCI-779, RAD 001, or AP23573 and the EGFR inhibitor is ZD-1839, OSI-774, PD-153053, PD-168393, IMC-C225 or CI-1033.

In typical methods, the expression of the biomarker polypeptides is examined using an antibody such as an antibody that binds an epitope comprising a phosphorylated serine residue at position 235 in SEQ ID NO: 1, an antibody that binds an epitope comprising a phosphorylated serine residue at position 473 in SEQ ID NO: 4, or an antibody that binds an epitope comprising a phosphorylated threonine residue at position 202 and tyrosine 204 in SEQ ID NO: 8. Optionally, the sample is a paraffin embedded biopsy sample.

Another embodiment of the invention is a method for identifying a mammalian tumor that does not express a PTEN polypeptide (SEQ ID NO: 5) and which is not likely to respond or is nonresponsive to an inhibitor of mTOR polypeptide (SEQ ID NO: 2) activity, the method comprising examining a sample obtained from the tumor for the presence of phosphorylated S6 ribosomal polypeptide (SEQ ID NO: 1) after contacting the tumor or the sample with the inhibitor, wherein, an observable decrease in phosphorylated S6 ribosomal polypeptide in the sample, as compared to a control that is not contacted with the inhibitor identifies the tumor as likely to respond or responsive to the inhibitor, and wherein no observable decrease in phosphorylated S6 ribosomal polypeptide in the sample, as compared to a control identifies the tumor as not likely to respond or nonresponsive to the inhibitor.

Yet another embodiment of the invention is a method for identifying a mammalian tumor that expresses a PTEN polypeptide (SEQ ID NO: 5) and which is not likely to respond or is nonresponsive to an inhibitor of EGFR polypeptide (SEQ ID NO: 7) activity, the method comprising examining a sample obtained from the tumor for the presence of EGFR (SEQ ID NO: 7) and the presence of a phosphorylated AKT polypeptide (SEQ ID NO: 4) or the presence of a phosphorylated ERK polypeptide (SEQ ID NO: 8) after contacting the tumor or the sample with the inhibitor, wherein an increase in the levels of the EGFR polypeptide and the levels of phosphorylated AKT polypeptide or phosphorylated ERK polypeptide identifies the tumor as not likely to respond or nonresponsive to the inhibitor. Optionally, the sample obtained from the tumor is examined for the presence of a phosphorylated AKT polypeptide (SEQ ID NO: 4) and the presence of a phosphorylated ERK polypeptide (SEQ ID NO: 8).

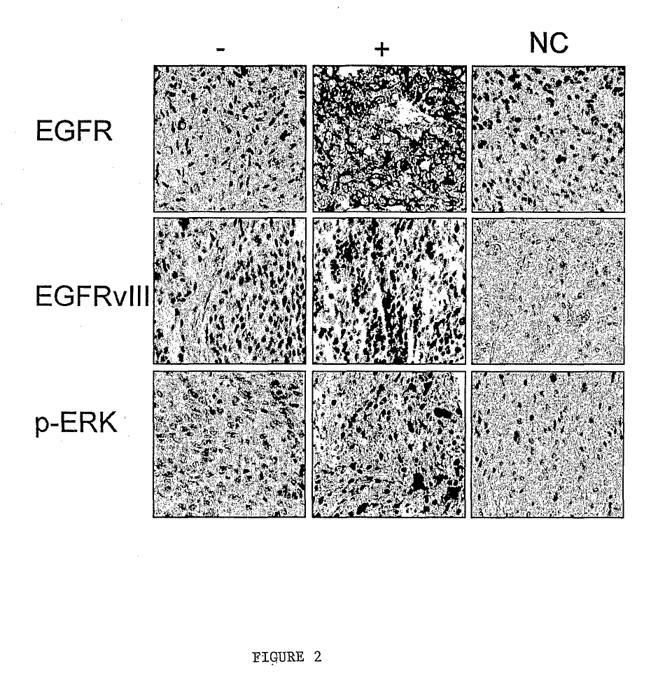

As noted above, certain embodiments of the invention include the examination of the expression of a polypeptide or phosphorylation of a polypeptide. As is known in the art, the examination of such polypeptide expression and polypeptide phosphorylation status in a cell or tissue sample is typically evaluated as compared to a control, i.e. a control cell and/or tissue sample that has a defined or predetermined level of polypeptide expression or phosphorylation. In an example of polypeptide phosphorylation, a control can be a normal tissue (e.g. non cancerous glial cells) where it is observed that a polypeptide is typically not phosphorylated. In an example of polypeptide expression, Example 3 and FIG. 2 provided illustrative examples of the methods of the invention using such controls, in particular, a PTEN expression grading system known the art that uses vascular endothelium as a control. Specifically PTEN immunohistochemical staining (which is directly correlated with PTEN expression) is scored according to an established scale of 0-2, in which the vascular endothelium (score of 2) serves as an internal control. Tumor cells are graded as 2 if their staining intensity is equal to that of the vascular endothelium, 1 if it is diminished relative to the vascular endothelium, and 0 if it is undetectable in the tumor cells and present in the vascular endothelium. This scoring system, which has been shown to be highly consistent between different cancer cell types, including gliomas (as disclosed herein) and cancers of the breast, ovary, pancreas and colon, allows artisans to readily examine the expression levels of PTEN polypeptides in a sample such as a formalin fixed, paraffin embedded biopsy sample.

Additional embodiments of the invention include a method for identifying a mammalian cancer cell that does not express a PTEN polypeptide (SEQ ID NO: 5) and which is likely to exhibit growth inhibition when contacted with an inhibitor of mTOR polypeptide (SEQ ID NO: 2) activity, the method comprising examining the cancer cell for the presence of phosphorylated S6 ribosomal polypeptide (SEQ ID NO: 1) after contacting the cancer cell with the inhibitor, wherein, an observable decrease in phosphorylated S6 ribosomal polypeptide in the sample, as compared to a control mammalian cancer cell that is not contacted with the inhibitor identifies the cancer cell as likely to exhibit growth inhibition when contacted with the inhibitor, and further wherein no observable decrease in phosphorylated S6 ribosomal polypeptide in the sample, as compared to a control mammalian cell identifies the cancer cell as not likely to exhibit growth inhibition when contacted with the inhibitor. In these methods, the inhibitor of mTOR polypeptide activity is optionally rapamycin, CCI-779, RAD 001, or AP23573. Typically, the expression of the PTEN polypeptide or the presence of phosphorylated S6 ribosomal polypeptide is examined using an antibody that binds the PTEN polypeptide or the phosphorylated S6 ribosomal polypeptide (e.g. an antibody that binds an epitope comprising a phosphorylated serine residue at position 235 in SEQ ID NO: 1). Preferably, the mammalian cancer cell is obtained from a paraffin embedded biopsy sample.

Another embodiment of the invention is a method for identifying a mammalian cancer cell that expresses a PTEN polypeptide (SEQ ID NO: 5) and which is not likely to exhibit growth inhibition when contacted with inhibitor of EGFR polypeptide (SEQ ID NO: 7) activity, the method comprising examining the cancer cell for the presence of EGFR (SEQ ID NO: 7), the presence of a phosphorylated AKT polypeptide (SEQ ID NO: 4) or a the presence of a phosphorylated ERK polypeptide (SEQ ID NO: 8), wherein an increase in the levels of the EGFR polypeptide and the levels of phosphorylated AKT polypeptide or phosphorylated ERK polypeptide identifies the cancer cell as not likely to exhibit growth inhibition when contacted with inhibitor of the EGFR polypeptide. In these methods, the inhibitor of EGFR activity is optionally ZD-1839, OSI-774, PD-153053, PD-168393 or CI-1033. Typically, the expression of the PTEN polypeptide or the presence of EGFR polypeptide is examined using an antibody that binds the PTEN polypeptide or the EGFR polypeptide. Optionally, the presence of phosphorylated AKT is examined using an antibody that binds an epitope comprising a phosphorylated serine residue at position 473 in SEQ ID NO: 4 and the presence of phosphorylated ERK is examined using an antibody that binds an epitope comprising a phosphorylated threonine residue at position 202 or a phosphorylated tyrosine residue at position 204 in SEQ ID NO: 8. In illustrative methods, the mammalian cancer cell is obtained from a paraffin embedded biopsy sample.

Another embodiment of the invention is a method for determining the responsiveness of a mammalian cancer cell to a growth inhibitory agent selected from the group consisting of a EGFR polypeptide (SEQ ID NO: 7) inhibitor or an mTOR polypeptide (SEQ ID NO: 2) inhibitor, the method comprising examining the glioblastoma cell for the presence of a S6 polypeptide (SEQ ID NO: 1) having a phosphorylated serine, threonine or tyrosine residue; a mTOR polypeptide (SEQ ID NO: 2) having a phosphorylated serine, threonine or tyrosine residue; a FKHR polypeptide (SEQ ID NO: 3) having a phosphorylated serine, threonine or tyrosine residue; a AKT polypeptide (SEQ ID NO: 4) having a phosphorylated serine, threonine or tyrosine residue; a ERK polypeptide (SEQ ID NO: 8) having a phosphorylated serine, threonine or tyrosine residue; or the expression of the PTEN polypeptide (SEQ ID NO: 5), wherein the presence of a phosphorylated S6, mTOR, FKHR, AKT or ERK polypeptide, or decreased levels of expression of the PTEN polypeptide in the glioblastoma cell relative to a control mammalian vascular endothelial cell determines the responsiveness of the mammalian cancer cell to the growth inhibitory agent. Optionally in such methods, the mammalian cancer cell has been contacted with the growth inhibitory agent. Alternatively, the mammalian cancer cell has not been contacted with the growth inhibitory agent.

Yet another embodiment of the invention is a method to obtain information used to identify a therapeutic agent for treating glioblastoma in a human, the method comprising examining a cancer cell obtained from the human for the presence of: a S6 polypeptide (SEQ ID NO: 1) having a phosphorylated serine, threonine or tyrosine residue; a mTOR polypeptide (SEQ ID NO: 2) having a phosphorylated serine, threonine or tyrosine residue; a FKHR polypeptide (SEQ ID NO: 3) having a phosphorylated serine, threonine or tyrosine residue; a AKT polypeptide (SEQ ID NO: 4) having a phosphorylated serine, threonine or tyrosine residue; or decreased levels of expression of the PTEN polypeptide (SEQ ID NO: 5), wherein the presence of a phosphorylated S6, mTOR, FKHR or AKT polypeptide, or decreased levels of expression of the PTEN polypeptide in the cancer cell provides information used to identify a therapeutic agent for treating the cancer in the human. Optionally in this method, the cancer cell is examined for the presence of a plurality of these characteristics. In one such embodiment, the cancer cell is examined for the presence of a S6 polypeptide (SEQ ID NO: 1) having a phosphorylated serine, threonine or tyrosine residue and decreased levels of expression of the PTEN polypeptide (SEQ ID NO: 5). Optionally the cancer cell is in a paraffin embedded biopsy sample.

As noted above, embodiments of the invention typically utilize antibodies that specifically bind phosphorylated polypeptides, i.e. polypeptides having a phosphorylated serine, threonine or tyrosine residue. In this context the disclosure provides antibodies that bind to specific epitopes comprising a phosphorylated residue (e.g. serine at position 2481 in SEQ ID NO: 2). By utilizing antibodies that bind to an epitope that comprises a phosphorylated residue (i.e. phospho-specific antibodies) but which do not bind to the unphosphorylated form of the same polypeptide, these phospho-specific antibodies can be used to examine the activation status of a pathway, where the activation is associated with phosphorylation of one or more specified residues. In certain embodiments of the invention, the phosphorylation status and/or expression levels of multiple members of a signalling pathway (e.g. S6 and mTOR) are examined as a confirmatory assessment of the signalling cascade associated with the pathway.

Certain embodiments of the invention are used with formalin fixed, paraffin embedded biopsy samples. In particular, the disclosure provided herein demonstrates that antibodies such as phospho-specific antibodies can be used with antigen samples processed in this manner. Significantly, the disclosure provided herein further demonstrates that the methods using these samples provide an accurate demonstration of the physiological status of the pathways in these samples. Consequently, the disclosure provided herein demonstrates how the methods of the invention are well suited for use with commonly available clinical samples.

In one illustrative embodiment of the invention, the presence of a S6 polypeptide (SEQ ID NO: 1) having a phosphorylated serine, threonine or tyrosine residue is examined using an antibody that binds an epitope comprising a phosphorylated serine residue at position 235 in SEQ ID NO: 1. In another illustrative embodiment of the invention, the presence of a mTOR polypeptide (SEQ ID NO: 2) having a phosphorylated serine, threonine or tyrosine residue is examined using an antibody that binds an epitope comprising a phosphorylated serine residue at position 2481 in SEQ ID NO: 2. In another illustrative embodiment of the invention, the presence of a FKHR polypeptide (SEQ ID NO: 3) having a phosphorylated serine, threonine or tyrosine residue is examined using an antibody that binds an epitope comprising a phosphorylated threonine residue at position 24 in SEQ ID NO: 3. In another illustrative embodiment of the invention, the presence of a AKT polypeptide (SEQ ID NO: 4) having a phosphorylated serine, threonine or tyrosine residue is examined using an antibody that binds an epitope comprising a phosphorylated serine residue at position 473 in SEQ ID NO: 4. The expression levels and/or phosphorylation of additional polypeptide markers can also be examined. Illustrative example of such additional markers include Ki-67 (SEQ ID NO: 9) and p-H3 histone H3 (SEQ ID NO: 10).

Yet another embodiment of the invention is a method of examining a mammalian cell for evidence of Akt pathway activation comprising examining the mammalian cell for the presence of: a S6 polypeptide, (SEQ ID NO: 1) having a phosphorylated serine residue at position 235 in SEQ ID NO: 1; a mTOR polypeptide (SEQ ID NO: 2) having a phosphorylated serine residue at position 2481 in SEQ ID NO: 2; a FKHR polypeptide (SEQ ID NO: 3) having a phosphorylated threonine residue at position 24 in SEQ ID NO: 3; a AKT polypeptide (SEQ ID NO: 4) having a phosphorylated serine residue at position 473 in SEQ ID NO: 4; or decreased levels of expression of the PTEN polypeptide (SEQ ID NO: 5), wherein the presence of a phosphorylated S6, mTOR, FKHR or AKT polypeptide, or decreased levels of expression of the PTEN polypeptide evidence of Akt pathway activation in the mammalian cell. Optionally the mammalian cell is examined for the presence of a plurality of characteristics such as a S6 polypeptide (SEQ ID NO: 1) having a phosphorylated serine residue at position 235 in SEQ ID NO: 1 and decreased levels of expression of the PTEN polypeptide (SEQ ID NO: 5). Typically in this method, the mammalian cell is a cancer cell such as a cancer cell is of a glioblastoma lineage.

Certain embodiments of the invention comprise further methodological steps, for example using the results of the examination in a prognostic determination of tumor progression and/or using the results of the examination to identify the presence of a glioblastoma characterized by a short time from initial diagnosis to patient death. Optionally the further methodological steps include the step of using the results of the examination to identify a therapeutic agent for treating the glioblastoma such as the step of using the results of the examination to evaluate the effect of rapamycin on the glioblastoma cancer cell. Optionally the mammalian cell is in a paraffin embedded biopsy sample.

A preferred embodiment of the invention is a method of examining a mammalian cell for evidence of Akt pathway activation comprising using a phospho-specific antibody to examine the cell for the presence of a phosphorylated protein in the mammalian cell selected from the group consisting of mTOR, FKHR and S6, wherein the presence of a phosphorylated mTOR, FKHR or S6 protein in the mammalian cell provides evidence of Akt pathway activation. In highly preferred embodiments, the cell is examined for the concurrent phosphorylation of mTOR, FKHR S6 proteins. Such methods typically include an optional step of using a phospho-specific antibody to examine the cell for evidence of phosphorylation of a Akt protein in the mammalian cell. In such methods, the mammalian cell is typically a cancer cell that is present in a paraffin embedded biopsy sample. In highly preferred embodiments of the invention the cancer cell is of the glioblastoma lineage.

Yet another embodiment of the invention is a method of examining a mammalian cell for evidence of Erk pathway activation comprising using a phospho-specific antibody to examine the cell for presence of phosphorylated p-44/42 MAP kinase proteins in the cells, wherein the presence of phosphorylated p-44/42 MAP kinase proteins in the mammalian cell provides evidence of Erk pathway activation. In preferred methods, the mammalian cell is present in a paraffin embedded biopsy sample obtained from an individual suspected of suffering from glioblastoma.

Another embodiment of the invention is a method of examining a tissue sample for the presence of mammalian glioblastoma cells having a phenotype characterized by a shorter time to tumor progression comprising using phospho-specific antibodies to examine the cell for the presence of phosphorylated mTOR, FKHR and S6 proteins in the cells, wherein the presence of a phosphorylated mTOR, FKHR and S6 proteins in the mammalian cell provides evidence of the phenotype. A related embodiment of the invention is a method of examining a tissue sample for the presence of mammalian glioblastoma cells having a phenotype characterized by a short time from initial diagnosis to patient death comprising using phospho-specific antibodies to examine the cell for the presence of phosphorylated mTOR, FKHR and S6 proteins in the cells, wherein the presence of a phosphorylated mTOR, FKHR and S6 proteins in the mammalian cell provides evidence of the phenotype.

Another embodiment of the invention is a method of examining a tissue sample for the presence of mammalian glioblastoma cells having a phenotype characterized by a shorter time to tumor progression comprising using a phospho-specific antibody to examine the cell for the presence of phosphorylated Erk proteins in the cells, wherein the presence of a phosphorylated Erk proteins in the mammalian cell provides evidence of the phenotype. A related embodiment of the invention is a method of examining a tissue sample for the presence of mammalian glioblastoma cells having a phenotype characterized by a short time from initial diagnosis to patient death comprising using phospho-specific antibodies to examine the cell for the presence of phosphorylated p-44/42 MAP kinase proteins in the cells, wherein the presence of a phosphorylated p-44/42 MAP kinase proteins in the mammalian cell provides evidence of the phenotype.

Yet another embodiment of the invention is a method of obtaining information useful for identifying an appropriate therapeutic agent to use to treat an individual suffering from glioblastoma comprising examining a tissue sample from the patient for the presence of glioblastoma cells having a phosphorylated protein selected from the group consisting of mTOR, FKHR and S6, wherein the presence of a phosphorylated mTOR, FKHR or S6 protein in the mammalian cell provides information useful for identifying an appropriate therapeutic agent to use to treat an individual suffering from glioblastoma. In preferred embodiments of the invention the mammalian cell is examined for the presence of at least two and more preferably three phosphorylated proteins selected from the group consisting of mTOR, FKHR and S6. Typically the therapeutic agent is a kinase inhibitor of the Akt pathway.

Another embodiment of the invention is a method of obtaining information useful for identifying an appropriate therapeutic agent to use to treat an individual suffering from glioblastoma comprising examining a tissue sample from the patient for the presence of glioblastoma cells having phosphorylated Erk proteins, wherein the presence of phosphorylated Erk proteins in the mammalian cell provides information that can be used to identify an appropriate therapeutic agent to use to treat an individual suffering from glioblastoma.

Another embodiment of the invention is a method of examining a mammalian cell for evidence of Akt pathway activation comprising examining the cell for the expression of the EGFR and the EGFRvIII proteins, wherein the coexpression of the EGFR and the EGFRvIII proteins in the cell provides evidence of Akt pathway activation. A related embodiment of the invention is a method of examining a mammalian cell for evidence of Erk pathway activation comprising examining the cell for the expression of the EGFR and the EGFRvIII proteins, wherein the coexpression of the EGFR and the EGFRvIII proteins in the cell provides evidence of Erk pathway activation. Yet another embodiment of the invention is a method of examining a mammalian glioblastoma cell for evidence of Akt pathway activation, wherein the mammalian glioblastoma cell is obtained from a paraffin embedded biopsy sample, the method comprising examining the cell for decreased expression of the PTEN protein, wherein a decrease in the expression of the PTEN protein cell provides evidence of Akt pathway activation.

### Articles of Manufacture of the Invention

Embodiments of the invention also include articles of manufacture and/or kits designed to facilitate the methods of the invention. Typically such kits include instructions for using the elements therein according to the methods of the present invention. Such kits can comprise a carrier means being compartmentalized to receive in close confinement one or more container means such as vials, tubes, and the like, each of the container means comprising one of the separate elements to be used in the method. For example, one of the container means can comprise one or more of the antibodies disclosed herein (an anti-S6 antibody for example) that is or can be detectably labeled with a marker. For kits utilizes immunological methods (e.g. immunohistochemistry and Western blotting) to detect the target proteins, the kit can also have containers containing buffers for these methods and/or containers comprising antibodies labelled with a reporter-means, such as a chromophore or radioactive molecule. In addition, for kits which utilize additional methodologies such as caspase-3 assays or tunnel assays of apoptosis, additional reagents associated with these techniques can be further included in the kits.

In a typical embodiment of the invention, an article of manufacture containing materials useful for the examination of the disorders described above is provided. The article of manufacture comprises a container and a label. Suitable containers include, for example, bottles, vials, syringes, and test tubes. The containers may be formed from a variety of materials such as glass or plastic. The container can hold a composition (e.g. an antibody composition) which is effective for examining mammalian cells (e.g. prostate cancer or glioblastoma cells). The label on, or associated with, the container indicates that the composition is used for examining cellular polypeptides. The article of manufacture may further comprise a second container comprising a buffer, such as phosphate-buffeted saline, Ringer's solution and dextrose solution. It may further include other materials desirable from a commercial and user standpoint, including other buffets, diluents, filters, needles, syringes, and package inserts with instructions for use.

One such embodiment of the invention is a kit comprising at least one antibody selected from the group consisting of: an antibody that binds a S6 polypeptide (SEQ ID NO: 1), wherein the S6 polypeptide epitope bound by the antibody comprises a phosphorylated serine, threonine or tyrosine residue; an antibody that binds a mTOR polypeptide (SEQ ID NO: 2), wherein the mTOR polypeptide epitope bound by the antibody comprises a phosphorylated serine, threonine or tyrosine residue; an antibody that binds a FKHR polypeptide (SEQ ID NO: 3), wherein the FKHR polypeptide epitope bound by the antibody comprises a phosphorylated serine, threonine or tyrosine residue; and an antibody that binds a AKT polypeptide (SEQ ID NO: 4), wherein the AKT polypeptide epitope bound by the antibody comprises a phosphorylated serine, threonine or tyrosine residue; and wherein the kit further includes instructions for using the antibody to examining a mammalian cell for evidence of AKT pathway activation. Optionally the kit further comprises an antibody that binds a PTEN polypeptide (SEQ ID NO: 5). The kits of the invention can further include antibodies to additional polypeptides such as Ki-67 (SEQ ID NO: 9) and p-H3 histone H3 (SEQ ID NO: 10).

Another embodiment of the invention is a kit comprising an antibody capable of immunospecifically binding a phosphorylated protein in a mammalian cell selected from the group consisting of phosphorylated Akt, mTOR, FKHR and S6 proteins and instructions for using the antibody to examining the mammalian cell for evidence of Akt pathway activation. In preferred methods, the kit comprises different antibodies, each of which is capable of immunospecifically binding 2, 3 or 4 phosphorylated proteins in a mammalian cell selected from the group consisting of phosphorylated Akt, mTOR, FKHR and S6 proteins. Another embodiment of the invention is a kit comprising an antibody capable of immunospecifically binding a phosphorylated p-44/42 MAP kinase proteins in a mammalian glioblastoma cell present in a paraffin embedded biopsy sample and instructions for using the antibody to examining the mammalian cell for evidence of Erk pathway activation.

Yet another embodiment of the invention is a kit for characterizing a mammalian prostate cancer or glioblastoma (GBM) tumor or cell, the kit comprising: an antibody that binds PTEN (SEQ ID NO: 5) and at least on of the following: an antibody that binds phosphorylated S6 ribosomal protein (SEQ ID NO: 1); an antibody that binds EFGR (SEQ ID NO: 7); an antibody that binds phosphorylated AKT (SEQ ID NO: 4); and/or an antibody that binds phosphorylated ERK (SEQ ID NO: 8); and at least one secondary antibody that binds to the above noted primary antibodies. Optionally the kit comprises a plurality of these antibodies. In a specific embodiment, the kit includes an antibody specific for S6 ribosomal protein (SEQ ID NO: 1) having a phosphorylated serine residue at position 235 in SEQ ID NO: 1; an antibody specific for AKT (SEQ ID NO: 4) having a phosphorylated serine residue at position 473 in SEQ ID NO: 4; or an antibody specific for ERK (SEQ ID NO: 8) having a phosphorylated threonine residue at position 202 or a phosphorylated tyrosine residue at position 204 in SEQ ID NO: 8. Another embodiment of the invention is a kit for characterizing a mammalian prostate cancer or glioma tumor or cell, the kit comprising: an antibody that binds PTEN (SEQ ID NO: 5); an antibody that binds phosphorylated S6 ribosomal polypeptide (SEQ ID NO: 1); an antibody that binds EFGR (SEQ ID NO: 7); an antibody that binds phosphorylated AKT (SEQ ID NO: 4); an antibody that binds phosphorylated ERK (SEQ ID NO: 8). Typically the kit further comprises a secondary antibody which binds to one of the primary antibodies directed to these polypeptides. Optionally the kit comprises a plurality of antibodies such as an antibody specific for S6 ribosomal polypeptide (SEQ ID NO: 1) having a phosphorylated serine residue at position 235 in SEQ ID NO: 1, an antibody specific for AKT (SEQ ID NO: 4) having a phosphorylated serine residue at position 473 in SEQ ID NO: 4; or antibody specific for ERK having a phosphorylated threonine residue at position 202 and tyrosine 204 in SEQ ID NO: 8. Optionally the kit further includes an antibody that binds Ki-67 polypeptide (SEQ ID NO: 9), p-H3 histone polypeptide (SEQ ID NO: 10) or caspase-3 polypeptide (SEQ ID NO: 11).

### Typical Protocols Useful to the Practice of the Invention

The methods of the present invention typically utilize antibodies directed to polypeptides in the PI3K/Akt pathway. Illustrative antibody compositions useful in the present invention are anti-phosphoprotein antibodies characterized as containing antibody molecules that specifically immunoreacts with a phosphorylated form of a polypeptide associated with the PI3K/Akt pathway. The polypeptide may be for example, S6, mTOR, FKHR, AKT or PTEN. By “specifically immunoreacts”, it is meant that the antibody binds to the phosphorylated form of polypeptide (i.e. is phospho-specific) and does not bind to the unphosphorylated form of the same polypeptide. Consequently, the phosphorylation associated with pathway activation can be examined with such antibodies. Therefore, the antibodies of the invention can distinguish between the phosphorylated and unphosphorylated forms of a polypeptides associated with the PI3K/Akt pathway. Consequently, the phosphorylation associated with pathway activation can be examined with such antibodies. Typically the assays of the invention include immunohistochemical techniques using the antibodies disclosed herein. For example, a sample can be examined for the presence of a biochemical pathway associated phosphorylated polypeptide such as phosphorylated ERK by using an antibody that binds an epitope comprising a phosphorylated threonine residue at position 202 and tyrosine 204 in SEQ ID NO: 8.

### 1. Antibodies

The antibodies useful in the invention may comprise polyclonal antibodies, for example affinity purified polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the appropriate polypeptide epitopes (e.g. a S6 polypeptide (SEQ ID NO: 1) having a phosphorylated serine, threonine or tyrosine residue, a mTOR polypeptide (SEQ ID NO: 2) having a phosphorylated serine, threonine or tyrosine residue, a FKHR polypeptide (SEQ ID NO: 3) having a phosphorylated serine, threonine or tyrosine residue, a ERK polypeptide (SEQ ID NO: 8) having a phosphorylated serine, threonine or tyrosine residue, a AKT polypeptide (SEQ ID NO: 4) having a phosphorylated serine, threonine or tyrosine residue, or a PTEN polypeptide) or a fusion protein thereof.

In addition, it may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include a phosphorylated S6, mTOR, FKHR, ERK or AKT polypeptide or PTEN polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against phosphorylated S6, mTOR, FKHR, ERK or AKT polypeptides or PTEN and EGFR polypeptides. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640 medium. Alternatively, the hybridoma cells may be grown in vivo as ascites in a mammal. The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

Reactivity of antibodies with the cognate protein can be established by a number of well known means, including Western blot, immunoprecipitation, ELISA, and FACS analyses. A antibody or fragment thereof can be labeled with a detectable marker or conjugated to a second molecule. Suitable detectable markets include, but are not limited to, a radioisotope, a fluorescent compound, a bioluminescent compound, chemiluminescent compound, a metal chelator or an enzyme.

### 2. Assays

The invention provides assays for examining cellular pathways associated with disregulated cell growth. Certain embodiments of the invention include the steps of detecting the presence of phosphorylated S6, mTOR, FKHR, AKT or ERK polypeptides or PTEN and EGFR polypeptides in a tissue. Methods for detecting these polypeptides are well known and include, for example, immunoprecipitation, immunohistochemical analysis, Western blot analysis, molecular binding assays, ELISA, ELIFA and the like.

Typically the assays of the invention include immunohistochemical techniques. Immunohistochemical techniques as used herein encompasses the use of reagents detecting cell specific markets, such reagents include, for example antibodies. Antibodies, including monoclonal antibodies, polyclonal antibodies and fragments thereof, are often used to identify proteins or polypeptides of interest in a sample. A number of techniques are utilized to label objects of interest according to immunohistochemical techniques. Such techniques are discussed in Current Protocols in Molecular Biology, Unit 14 et seq., eds. Ausubel, et al., John Wiley & Sons, 1995, the disclosure of which is incorporated herein by reference. Typical protocols include staining a paraffin embedded tissue section prepared according to a conventional procedure (see, e.g. U.S. Pat. No. 6,631,203).

Certain embodiments of the invention include tunnel assays a markers of apoptosis. Typically, a TUNNEL assay is performed essentially as follows: the percentage of apoptotic cells are detected by the APO-BRDU terminal deoxynucleotidyl transferase (TdT)-mediated dUTP-biotin nick end-labeling assay (see, e.g. Gavrieli, et al., J. Cell Biol. 119: 493-501) according to manufacturer's instructions (see, e.g. Phoenix Flow Systems, Phoenix, Ariz.). For further discussions of TUNNEL assays useful in methods of the invention see, e.g. Prochazkova et al., Biotechniques 2003 September; 35(3):528-34; Duan et al., J Pathol. 2003 February; 199(2):221-8; and Walker et al., J Pathol. 2001 October; 195(3):275-6.

Certain embodiments of the invention include caspase-3 assays. Those skilled in the art will appreciate that the caspase-3 assay measures the activation of caspase-3 enzyme, a critical early event of apoptosis induced death (see, e.g. U.S. Patent Application No. 20020159996 and U.S. Pat. No. 6,346,607). For further discussions of TUNEL assays useful in methods of the invention see, e.g. Duan et al., J Pathol. 2003 February; 199(2):221-8; and Walker et al., J Pathol. 2001 October; 195(3):275-6.

Throughout this application, various publications are referenced. The disclosures of these publications are hereby incorporated by reference herein in their entireties.

### EXAMPLES

Examples 1-8 provide illustrative disclosure showing embodiments of the invention used to examine glioma cells. Example 9 provides an illustrative disclosure showing embodiments of the invention used to examine prostate cancer cells.

### Example 1

### Patient Selection and Construction of Tissue Microarray

All patients participating in this study gave informed consent prior to surgery, in accordance with UCLA Institutional Review Board Policies. Formalin-fixed, paraffin-embedded tissue blocks were taken from 45 patients diagnosed with a glioblastoma at initial surgical resection and treated by the UCLA neuro-oncologist. The diagnosis was confirmed independently by at least two Neuropathologists. None of the patients were treated prior to removal of the tumor. Three representative 0.6 mm cores were obtained from diagnostic areas of the tumor blocks from each of the primary GBM patients; two from geographically distinct regions of tumor and one from a region of normal brain tissue when available (approximately ⅔ of cases). The cores were then inserted into a grid pattern in a recipient paraffin block using a tissue arrayer. Five-micron sections were cut from the tissue array and immunohistochemistry was performed. Serial sections from the tissue array were used for immunohistochemical analysis. Four tumors had sufficient material on the tissue array for analysis of PTEN, EGFR and EGFRvIII, but lacked sufficient material for analysis of p-Akt, p-mTOR, p-S6, p-FKHR and p-Erk.

### Example 2

### Immunohistochemical Staining

Sections from the tissue microarray were stained with monoclonal antibodies to PTEN (clone 6H2.1, Cascade Bioscience, Winchester Mass.), EGFR (clone 31G7, Zymed, San Francisco, Calif.), EGFRvIII (clone L8A4, a generous gift from Dr. Darrell Bigner), and phosphorylation specific antibodies directed against p-Akt (set 473) p-FKHR (thr24)/p-FKHRL1 (thr32), p-mTOR (set 2481), p-S6 ribosomal protein (ser 235/236) and p-44/42 MAP kinase (p-Erk) (thr202/tyr204) (Cell Signaling Technologies, Beverly, Mass.). Sections were baked at 60° C. and de-paraffinized with xylenes and graded ethanols. Heat-induced antigen retrieval was used as follows: for p-Erk, p-Akt, p-mTOR, p-FKHR/FKHRL1 and p-s6, 0.01 M citrate buffer, pH 6 for 25 minutes in a pressure cooker; for PTEN, 0.01M citrate buffer, pH 6 for 16 minutes in a microwave oven; EGFR, pronase (0.03 g/ml 0f 0.05 M Tris buffer, pH 7.4) at 37° C. for 8 minutes and for EGFRvIII, 0.01 M citrate buffer, pH 6 for 25 minutes in a vegetable steamer. Endogenous peroxidase activity was quenched with 3% hydrogen peroxide in methanol. Primary antibodies (PTEN at 1:400, EGFR at 1:150, EGFRvIII at 1:400, p-Akt 1:50, p-mTOR 1:50; p-FKHR/FKHRL1 1:50, pS6 1:50 and p-ERK at 1:50) were diluted in Tris buffered saline with 0.1% Tween and applied for 16 hours at 4° C., followed by anti-mouse or anti-rabbit biotinylated immunoglobulins (Vector) at 1:100 dilution for one hour, and finally, avidin-biotin complex (Elite ABC, Vector) for one hour. Negative control slides received normal mouse serum (DAKO) as the primary antibody. Diaminobenzidine tetrahydrochloride was used as the enzyme substrate to visualize specific antibody localization for PTEN, EGFR and EGFRvIII; Vector NovaRed (Vector) was used for phospho-specific antibodies. Slides were counterstained with Harris hematoxylin.

### Example 3

### Scoring and Interpretation of Immunohistochemistry

PTEN—PTEN staining was scored according to a previously established scale of 0-2, in which the vascular endothelium (score of 2) serves as an internal control (see, e.g., Perren et al., Am J Pathol. 157: 1097-103, 2000; Perren et al., Am J Pathol. 155: 1253-60, 1999; Zhou et al., Am J Pathol. 161: 439-47, 2002; Gimm et al., Am J Pathol. 156: 1693-700, 2000). Tumor cells are graded as 2 if their staining intensity is equal to that of the vascular endothelium, 1 if it is diminished relative to the endothelium, and 0 if it is undetectable in the tumor cells and present in the vascular endothelium (see, e.g., Zhou et al., Am J Pathol. 161: 439-47, 2002). This scoring system has been shown to be highly consistent between different cancer cell types, including breast (see, e.g., Perren et al., Am J Pathol. 155: 1253-60, 1999), ovarian (see, e.g., Mutter et al., Cancer Res. 61: 4311-4314, 2001), pancreas (see, e.g., Perren et al., Am J Pathol. 157: 1097-103, 2000) and colon (see, e.g., Zhou et al., Am J Pathol. 161: 439-47, 2002). Two Neuropathologists scored the tumors independently. In addition, tumors were scored by one of the Neuropathologists on two independent occasions. Both the inter-rater, and the intra-rater agreement were greater than 90%.

EGFR and EGFRvIII—Tumors demonstrating strong EGFR immunopositivity in greater than 20% of tumor cells were considered to be positive (see, e.g., Liotta et al., Jama. 286: 2211-4, 2001); tumors demonstrating at least focal moderate to strong immunoreactivity for EGFRvIII were considered positive, as previously reported (see, e.g., Choe et al., Clin Cancer Res. 8: 2894-901, 2002). The inter-rater and intra-rater agreement for EGFR and EGFRvIII were >90%.

Phosphorylation specific antibodies—Phospho-Akt, mTOR, S6 and FKHR were scored on a scale of 0-2 (0+ no staining, 1+=mild intensity cytoplasmic staining, and 2+=strong cytoplasmic staining; staining of 1+ and 2+ were considered positive. The agreement between reviewers, as well as for the same reviewer on independent reviews, was 80% for p-Akt. It was higher for phosphorylated mTOR, S6 and FKHR, ranging from 87% for in TOR to 100% for S6. For phospho-ERK, tumors that focally contained greater than 5% positive nuclear staining were considered positive, as previously reported (see, e.g., Choe et al., Clin Cancer Res. 8: 2894-901, 2002). The agreement between reviewers, and for the same reviewer on independent reviews, was >85%.

### Example 4

### Statistical Analysis

The association between markers was analyzed using Fisher's Exact test. The software was available on the Simple Interactive Statistical Website which can be identified with a internet search using the terms “home.clara.net” (http://home.clara.net/sisa/index.htm). For analysis of prognostic factors, we excluded 13 patients who did not receive therapy other than surgery. These patients had a poor performance status at the time of diagnosis and elected not to have further therapy. All other patients had received at least standard involved field fractionated radiation therapy. Kaplan-Meier curves were generated to assess the association of variables with time from initial diagnosis to evidence of progression by imaging or clinical features (time to tumor progression) and time from initial diagnosis to death (overall survival). To identify statistically significant differences in time to progression and overall survival, the Wilcoxon two sample test was used.

### Example 5

### Assessment of PTEN/Akt Pathway by IHC

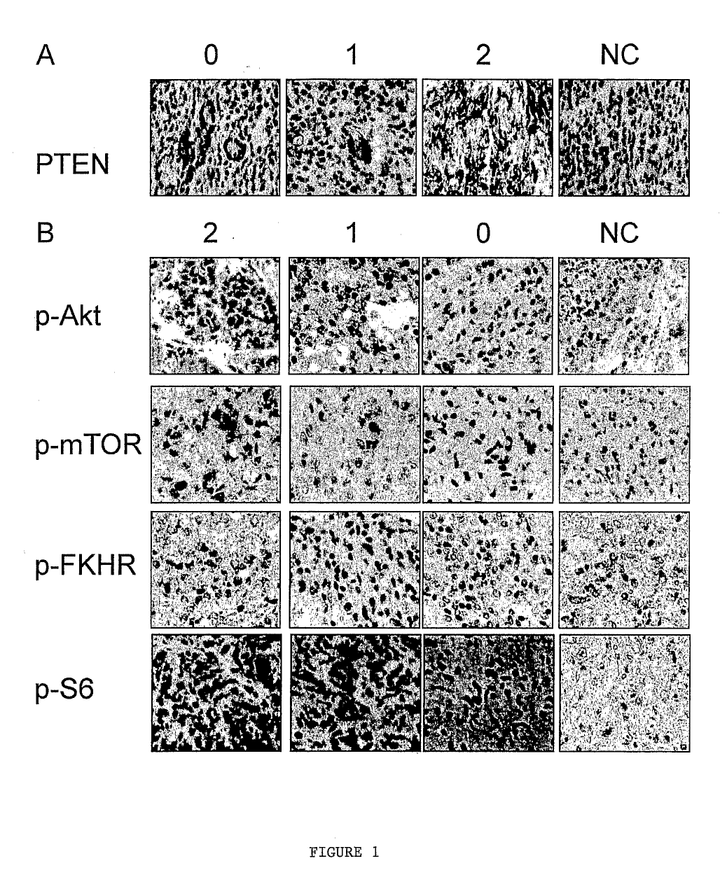

We constructed a tissue microarray consisting of samples from 45 untreated primary GBM patients (Table 1). All of the tumors presented as de novo grade IV tumors (“primary GBMs”) (see, e.g., Kleihues et al., Neuro-oncol. 1: 44-51, 1999). None of the patients received any radiation or chemotherapy prior to surgical resection. We focused on primary GBMs because they have a high incidence of PTEN mutations and EGFR over-expression (see, e.g., Kleihues et al., Neuro-oncol. 1: 44-51, 1999) and because this enabled us to analyze PI3′K/Akt pathway activation in the absence of prior therapy. The patients ranged in age from 28 to 88 with a median age of 58 (Table 1); all were diagnosed with a GBM on biopsy by at lease two independent Neuropathologists. PTEN protein expression was diminished or lost in 17/45 GBMs (38%) (FIG. 1, Table 2). This is in agreement with previous studies that have used DNA-based methods to detect PTEN loss in 30-40% of GBMs (see, e.g., Liu et al., Cancer Res. 57: 5254-7, 1997; Schmidt et al., J Neuropathol Exp Neurol. 58: 1170-83, 1999; Smith et al., J Natl Cancer Inst. 93: 1246-56, 2001). Akt phosphorylation was significantly associated with diminished PTEN immunohistochemical expression (p<0.00001) (FIG. 1, Table 2). PTEN loss was not significantly associated with expression of p-Erk (Table 2), whose activation is independent of PI3′K/Akt signaling. To determine whether Akt activation correlated with concurrent activation of downstream effectors, we used phosphorylation specific antibodies directed against mTOR, FKHR and S6. mTOR and FKHR are directly phosphorylated by Akt (see, e.g., Vivanco et al., Nat Rev Cancer. 2: 489-501, 2002; Hidalgo et al., Oncogene. 19: 6680-6686, 2000); S6 is phosphorylated by p70 S6 kinase, which is itself a target of Akt (see, e.g., Blume-Jensen et al., Nature. 411: 355-365, 2001). Akt activation was significantly associated with expression of p-mTOR (p=0.04) and p-FKHR (p=0.006) (Table 3). Akt activation was also correlated with strong S6 phosphorylation (2+) (p=0.001), although weaker S6 phosphorylation (1+) was also detected in Akt− negative tumors (Table 3). This latter result is not surprising considering that S6 can be activated by Erk in a PI3′K/Akt independent fashion (see, e.g., Iijima et al., J Biol Chem. 277: 23065-75, 2002; Shi et al., J Biol Chem. 277: 15712-20, 2002). Taken together, these results provides evidence that PI3′K/Akt pathway activation can be detected in routinely processed paraffin-embedded biopsy samples, and demonstrate that PTEN protein loss is associated with PI3′K/Akt pathway activation in GBMs.

### Example 6

### Akt Pathway Activation in GBMs Lacking PTEN Protein Loss: Assessment of EGFR/EGFRvIII-Mediated Signaling

PTEN loss did not appear to be the only route to Akt activation; expression of p-Akt and downstream effectors p-mTOR, p-FKHR and p-S6 was also detected in 28% of GBMs with no immunohistochemical PTEN loss (Table 2). Because the PI3′K/Akt pathway can be activated by EGFR signaling, we analyzed EGFR and EGFRvIII expression and assessed their association with PI3′K/Akt pathway activation in the setting of normal PTEN immunohistochemical staining. EGFR immunopositivity was detected in 60% of GBMs (FIG. 2), in line with previous reports (see, e.g., Smith et al., J Natl Cancer Inst. 93: 1246-56, 2001; Watanabe et al., Brain Pathol. 6: 217-23; discussion 23-4, 1996; Ekstrand et al., Proc Natl Acad Sci USA. 89: 4309-13, 1992; Frederick et al., Cancer Res. 60: 1383-7, 2000; Hayashi et al., Brain Pathol. 7: 871-5, 1997; Nagane et al., Cancer Lett. 162 Suppl: S17-S21, 2001; Nishikawa et al., Proc Natl Acad Sci USA. 91: 7727-31, 1994; Wikstrand et al., Cancer Res. 57: 4130-40, 1997). Immunohistochemical expression of the constitutively active mutant EGFRvIII was detected in 56% of EGFR positive tumors (44% of tumors overall) (FIG. 2) (Smith et al., J Natl Cancer Inst. 93: 1246-56, 2001; Nagane et al., Cancer Lett. 162 Suppl: S17-S21, 2001; Nishikawa et al., Proc Natl Acad Sci USA. 91: 7727-31, 1994; Wikstrand et al., Cancer Res. 57: 4130-40, 1997). Strongly activated Akt (2+ staining) was not detectable in GBMs with normal PTEN immunohistochemical expression that lacked EGFR and EGFRvIII expression (Table 2). In contrast, 36% of GBMs with normal PTEN immunohistochemical expression that also co-expressed EGFR and EGFRvIII stained strongly for activated Akt (p=0.06) (Table 2). Although the subset of tumors was small, co-expression of EGFRvIII along with EGFR appeared to be required for strong Akt activation (2+ staining) (Table 2). These results provides evidence that co-expression of EGFR and EGFRvIII can promote Akt activation in GBMs with normal PTEN protein expression. Consistent with this, downstream activation of mTOR, S6 and FKHR were also significantly more likely to be strongly activated (2+ staining) in GBMs with normal PTEN expression when EGFR and EGFRvIII were co-expressed (p<0.002).

In addition to the Akt pathway, EGFR, and EGFRvIII can also activate Erk. Therefore, we determined whether Erk phosphorylation was associated with EGFR and EGFRvIII expression. Overall, Erk phosphorylation was detected in 51% of GBMs. More importantly, expression of phosphorylated Erk was significantly associated with EGFR expression (p=0.007) (FIG. 2; Table 4). Phosphorylated Erk was expressed in 75% of EGFR+/EGFRvIII negative GBMs and 88% of EGFR+/EGFRvIII positive GBMs.

### Example 7

### Prognostic Implications of Akt and Erk Pathway Activation

Previous studies have not shown a clear prognostic implications for PTEN loss, EGFR over-expression or EGFRvIII expression in GBMs. In line with this, we found no statistically significant association between PTEN protein loss, EGFR or EGFRvIII expression and either time to progression or overall survival. In contrast, coordinate pathway activation appeared to have prognostic implications. Expression of p-Akt was not significantly associated with survival or progression. In contrast, activation of the downstream pathway, as detected by concurrent phosphorylation of mTOR, FKHR and S6, was significantly associated with both a shorter time to progression (p=0.002) and a decreased overall survival (p=0.02). This finding may reflect a contribution from additional inputs downstream of Akt, such as Erk-mediated activation of S6 kinase and nutrient-mediated activation of mTOR. Alternatively, this panel of three phospho-specific antibodies may be a mote sensitive method to detect Akt pathway activation than a single phospho-Akt antibody alone. Erk activation was also significantly associated with more rapid progression and diminished overall survival in this subset of primary GBM patients (<0.04) (Table 5). these findings are the first demonstration that pathway activation has an impact on tumor progression in GBM patients.

### Example 8

### Kinase Inhibitors Akt and Erk Pathway Activation

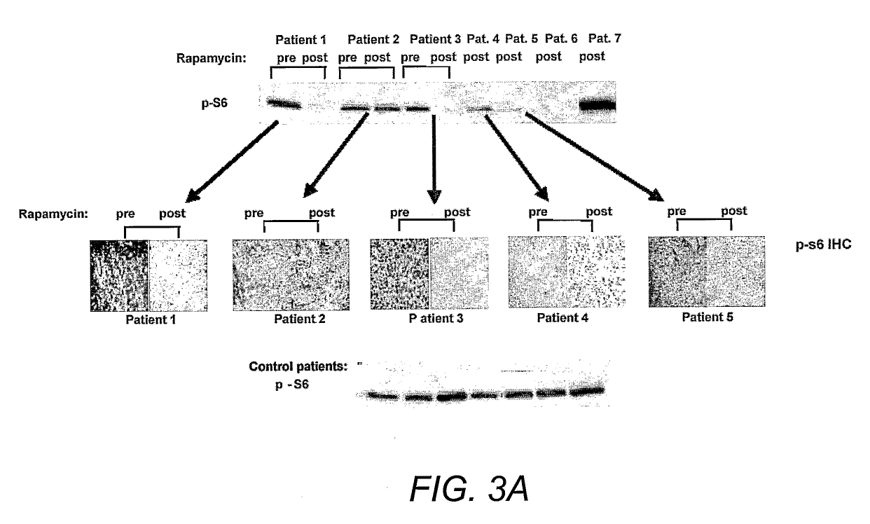

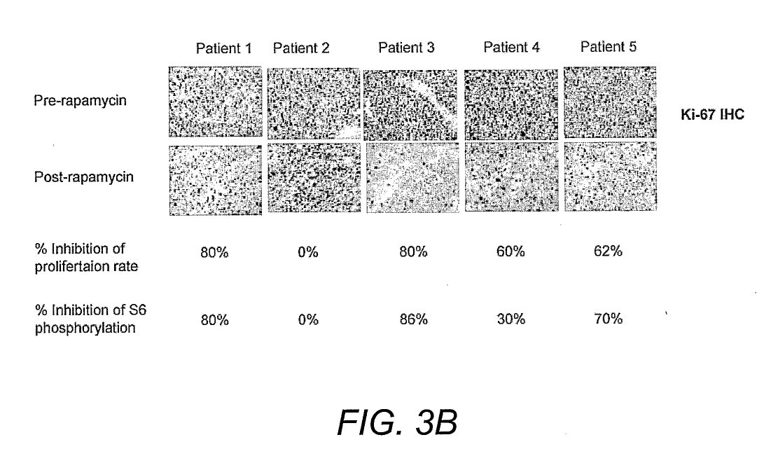

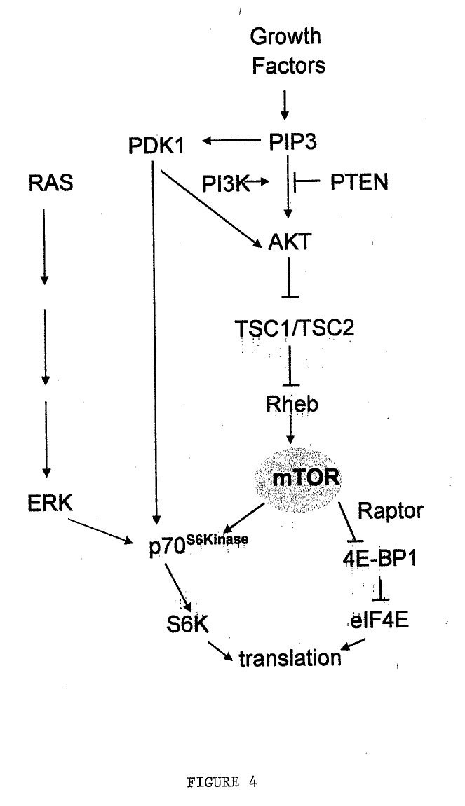

FIGS. 3A and 3B provide an illustration of the interaction between members of the PI3K/Akt pathway and kinase inhibitors.

FIG. 3A shows that rapamycin inhibits S6 phosphorylation in glioblastoma in vivo. In particular, FIG. 3A provides data from an analysis of a cohort of patients on a rapamycin clinical trial. This data shows that a substantial reduction in S6 phosphorylation relative to the initial biopsy was detected in the tumor in the majority of patients treated with rapamycin for 5 days prior to undergoing surgical resection. Control patients showed a uniformly high level of S6 phosphorylation. This data provides evidence that rapamycin inhibited mTOR signaling at the level of S6 phosphorylation in the majority of glioblastoma patients. In addition, this data illustrates how the detection of pathway activation by immunohistochemistry (IHC) correlates with detection by western blotting.

FIG. 3B shows that the rapamycin-mediated inhibition of S6 phosphorylation correlates with diminished tumor proliferation. In this Figure, Ki-67, a market of cellular proliferation was used to assess whether rapamycin-mediated inhibition of S6 had an effect on tumor growth. This data provides evidence that the rapamycin-mediated inhibition of mTOR signaling at the level of S6 phosphorylation correlated with diminished tumor cell proliferation.

### Example 9

### Antibody Based Profiling of the PI3K Pathway in Clinical Prostate Cancer

The following example illustrates the use of the methods and materials of the invention disclosed herein on prostate cancer samples. As noted above, constitutive activation of the PI3K pathway and its downstream effectors, as a result of PTEN loss or by other mechanisms, occurs in a high proportion of cancers such as prostate cancers, making it an ideal template for the design of clinical trials involving PI3K pathway inhibitors. Prostate cancers also present unique organ specific challenges, in that tumors are heterogeneous and the amount of diagnostic tissue is extremely limited. Working within these parameters, we disclose a set of immunohistochemical assays that define activation of the PI3K pathway in clinical samples. Using both univariate and multivariate analyses, we show that loss of PTEN is highly correlated with activation of AKT and this, in turn is associated with the phosphorylation of S6, one of its main effectors. Three antibodies directed at these targets define a molecular signature of PTEN loss and/or AKT pathway activation in prostate cancer.

We have previously shown that PTEN deficient prostate cancer cell lines and xenografts are more sensitive to the pharmacologic inhibition of mTOR using the rapamycin analogue, CCI-779, when compared to their wild type counterparts (Neshat et al., Proc Natl Acad Sci USA, 98: 10314-10319, 2001; Podsypanina et al., Proc Natl Acad Sci USA, 98: 10320-10325, 2001). We also demonstrated that the phosphorylation of S6 ribosomal protein (hereafter called S6) was a potential surrogate market for both pathway activation and mTOR inhibition by drug.

Lessons learned from molecularly targeted therapies point to two key elements that will ultimately determine how to efficiently translate these findings into the clinic (Druker et al., N Engl J Med, 344: 1031-1037, 2001; Druker et al., N Engl J Med, 344: 1038-1042, 2001; Bianco et al., Oncogene, 22: 2812-2822, 2003; Brognard et al., Cancer Res, 61: 3986-3997, 2001). One, we need to identify subsets of patients who will most likely to benefit from this class of drugs. The importance of this knowledge is exemplified by the recent experience with EGFR inhibitors in lung cancer, where knowing the mutation status of the EGFR can enrich for patients most likely to benefit from these class of drugs (Paez et al., Science, 304: 1497-1500, 2004; Lynch et al., N Engl J Med, 350: 2129-2139, 2004). Out current understanding suggests this may depend on the level of signal activation of the pathway being targeted and the molecular lesion leading to target activation. Second, we need to measure inhibition of the signaling pathway when using targeted therapy to guide dose selection and scheduling. Translating these goals to the clinic presents several challenges not encountered in vitro. Diagnostic needle core biopsies of the prostate invariably consist of small amounts of tissue. Also, unlike cancer cell lines, clinical samples of human prostate cancers are not homogenous populations, but consist instead of a mixture of both normal and tumor bearing cells. Clinical decisions using molecularly targeted agents are likely to require assessment of signaling pathway abnormalities using limited biopsy material.

With these goals in mind, this Example illustrates the detection of activated signaling pathways in situ on formalin fixed, paraffin embedded tissue, analogous to detecting Her-2/Neu amplification by immunohistochemistry in breast cancers. Since the target (mTOR) is a kinase, we examined the phosphorylation status of its downstream effectors by utilizing activation specific, phospho-specific antibodies. When examined within the context of a tissue microarray, we showed that PTEN loss was highly correlated with AKT activation, and AKT activation was correlated with upregulation of p-S6.

### Materials and Methods

### Human Prostate Cancer Tissue Microarray

We constructed a tissue microarray from formalin fixed, paraffin embedded archival tissue blocks, from 133 radical prostatectomies, performed for prostate cancer. The distribution of Gleason scores and Pathological TNM stage were as follows: 79 cases had Gleason score 5-6; 37 cases Gleason score 7 and 9 cases Gleason score 8-10. Eighty four cases were organ confined (i.e. Stage 2), 34 were Stage 3 and 13 cases were Stage 4. The array was designed so that three cores were taken from tumor bearing tissue and one core from morphologically benign tissue (only areas containing normal prostate glands and stroma were sampled; atrophic and hyperplastic glands were not included in the array construction or in the evaluation), from the same patient. These cores were placed adjacent to each other on the tissue array, enabling comparison of antibody staining pattern and intensity between benign and cancer tissue from the same patient.

### In Vitro Optimization of Phospho-Specific Antibodies

Cells (LNCaP, DU 145, HT 129) were grown in culture (DMEM supplemented with 10% FBS, L-Glutamine, Penicillin and Streptomycin) and exposed to vehicle, the PI3K inhibitor LY294002 (30 μM), the mTOR inhibitor, CCI-779 (20 nM) for 18 hours or the MEK inhibitor, UO126 (20 μM) combined with 100 ng/ml of EGF for 20 minutes. Cell pellets were prepared, formalin fixed and embedded in paraffin, then cut for immunohistochemical staining with phospho-AKT, -S6, -FKHR and -ERK antibodies. We performed antigen retrieval with a 10 mM sodium citrate buffer (pH 6.0) for 30 minutes in a pressure cooker. Peroxidase activity was quenched with 3% hydrogen peroxide in water. Sections were stained overnight at 4° C. with monoclonal antibodies to PTEN at 1:300 (clone 6H2.1; Cascade Bioscience, Winchester, Mass. This antibody recognizes a 99 amino acid epitope at the C-terminus of PTEN) and polyclonal phosphorylation-specific antibodies to p-AKT (Set 473), p-FKHR which recognizes phosphorylated forms of the forkhead family of transcription factors FKHR (FOXO 1), FKHRL1 (FOXO 3a) and AFX (FOXO 4), p-S6 Ribosomal protein (Set 235/236), p-ERK (Thr-202/Tyr-204) at 1:100 (Cell Signaling Technology, Beverly, Mass.). This was followed by application of biotinylated secondary antibodies (Vector) at 1:1000 dilution for 30 minutes and Avidin-biotin complex (Elite ABC; Vector). Negative control slides received normal mouse serum (DAKO) as the primary antibody. Diaminobenzidine tetrahydrochloride (DAB) was used as the enzyme substrate to visualize specific antibody localization for PTEN and p-AKT; Vector NovaRed (Vector) for p-FKHR, p-S6 and p-ERK. Slides were counterstained with Harris hematoxylin. For immunofluorescence assays, post fixation in paraformaldehyde, the cells were probed with Total AKT (1:50) and p-AKT (1:250); the secondary antibodies Alexa Flour anti-rabbit and anti-mouse (1:1000) [Molecular Probes, Eugene, Oreg.]. Following incubation, images were captured on a Nikon fluorescence microscope and then overlaid using Adobe Photoshop software.

### Scoring Protocols

### PTEN

PTEN staining was scored according to a previously established scale of 0-2, which has been shown to be highly consistent (Choe et al., Cancer Res, 63: 2742-2746, 2003; Gimm et al., Am J Pathol, 156: 1693-1700, 2000; Perren et al., Am J Pathol, 155: 1253-1260, 1999; Zhou et al., Am J Pathol, 157: 1123-1128, 2000). Tumor cells are graded as 2 if their staining intensity is equal to that of the adjacent benign cells, graded as 1 if their staining was diminished relative to the benign cells and 0 if staining intensity is undetectable in the tumor cells and is present in the benign cells. Only cores containing tumor and that were technically interpretable (e.g. immunoreactivity in the benign glands) were included in the scoring protocol. The arrays were scored by one genitourinary pathologist (G.V.T) on two occasions. In addition, twenty percent of the cases were randomly picked and scored by a second genitourinary pathologist in an independent manner, i.e. with no prior knowledge of the original results. Both inter-rater and intra-rater agreement were >90%.

### Phosphorylation Specific Antibodies

For scoring purposes, we only included membranous or cytoplasmic staining for p-AKT and only cytoplasmic staining for p-FKHR and p-S6. p-AKT, p-S6 and p-FKHR were scored according to a scale of 0-2 (0=no staining; 1=mild intensity of cytoplasmic staining and 2=strong cytoplasmic staining). For p-AKT and p-S6, staining of 1 and 2 were considered positive; for p-S6, staining of 2 was considered positive, as previously published (Choe et al., Cancer Res, 63: 2742-2746, 2003). For p-ERK, tumors that focally contained greater than 5% positive nuclear staining were considered positive, as previously reported (Choe et al., Cancer Res, 63: 2742-2746, 2003; Mukohara et al., Lung Cancer, 41: 123-130, 2003). The agreement between the pathologists (see PTEN scoring protocol) as well as the same pathologist on independent reviews was 85% for p-AKT and p-S6K; 80% for p-FKHR and 90% for p-ERK.

### Statistical Analysis of Immunohistochemical Scoring

When studying the correlations between the staining scores, we used Pearson correlation, which is appropriate for ordinally scored data (Table 7). When fitting a logistic regression model where a staining score was the outcome, we dichotomized the staining score (e.g. 0 vs. 1, 2), i.e. no expression versus at least some expression. The computation of the Pearson correlations and the logistic regression analysis were all carried with the R software (http://cran.r-project.org/) (see, e.g. Ihaka et al., J. Comput. Graphical Statistics, 5: 299-314, 1996). We then fitted two multivariate logistic regression models (Table 8). First we regressed PTEN status on p-AKT, p-FKHR and p-S6. Second we regressed p-AKT status on the p-FKHR family and p-S6K.

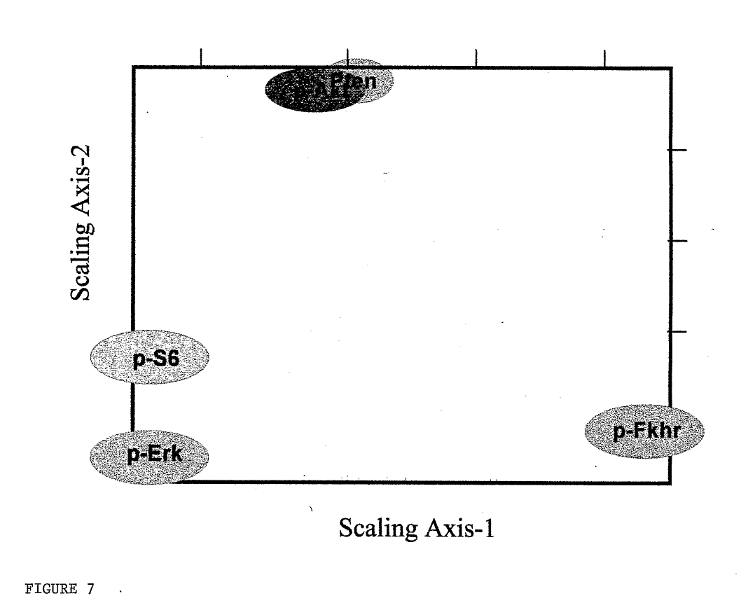

To depict the relationship between the variables, we used the R function cmd scale to arrive at a two-dimensional classical multidimensional scaling plot (FIG. 7). The aim of the MDS is to represent dissimilarities (here, one minus the squared Pearson correlation) between points (here, biomarker staining scores) well by choosing a two-dimensional configuration of points that minimizes a “stress” function (Cox et al., Multidimensional Scaling. United Kingdom: CRC Press, 2001).

### Characterization of PTEN and the Phospho-Specific Antibodies

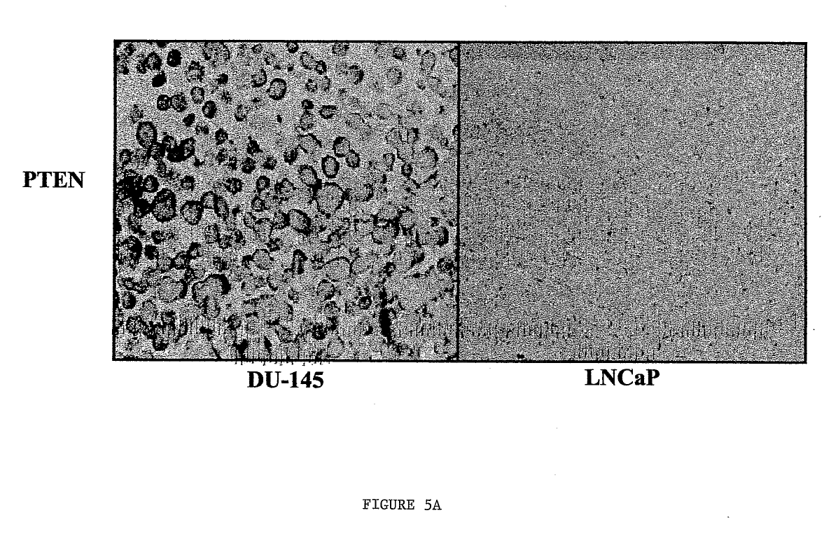

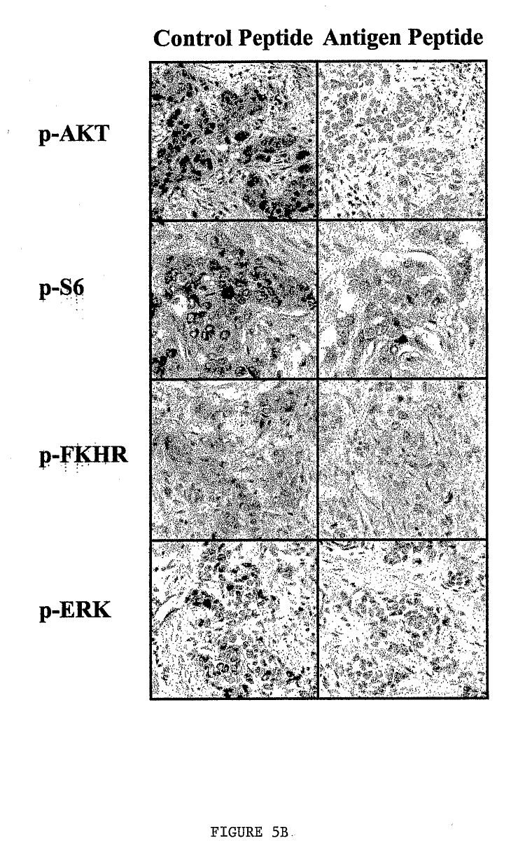

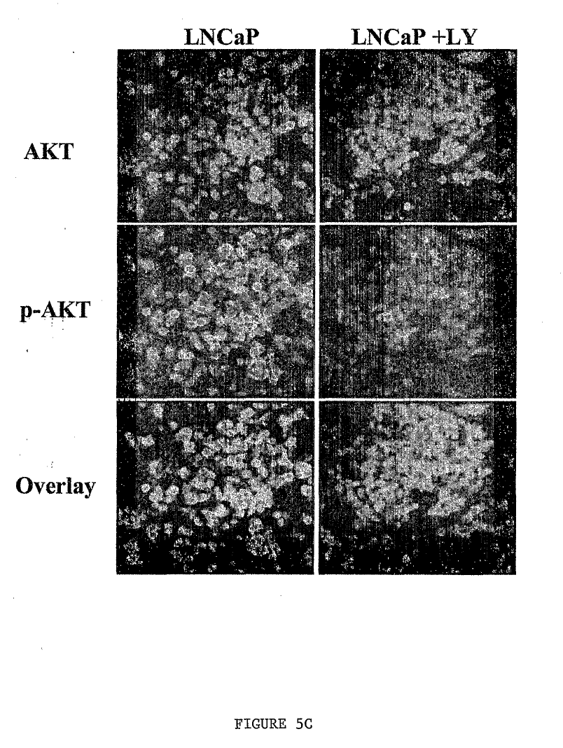

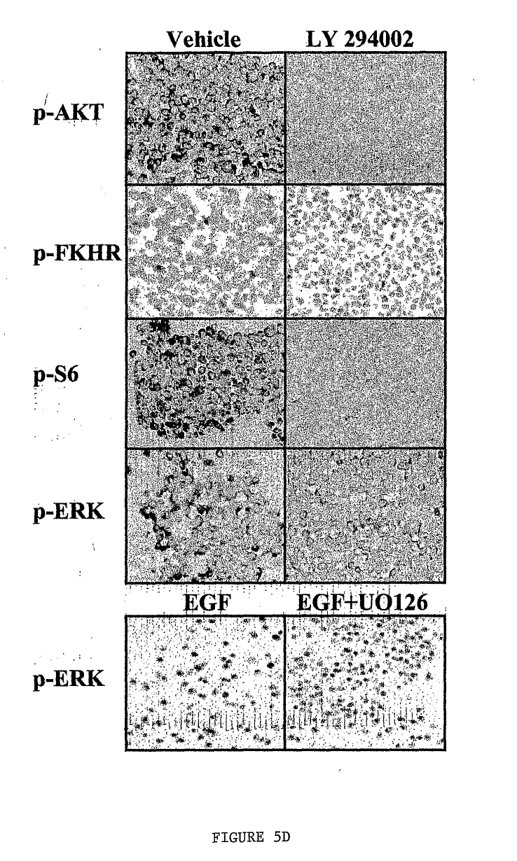

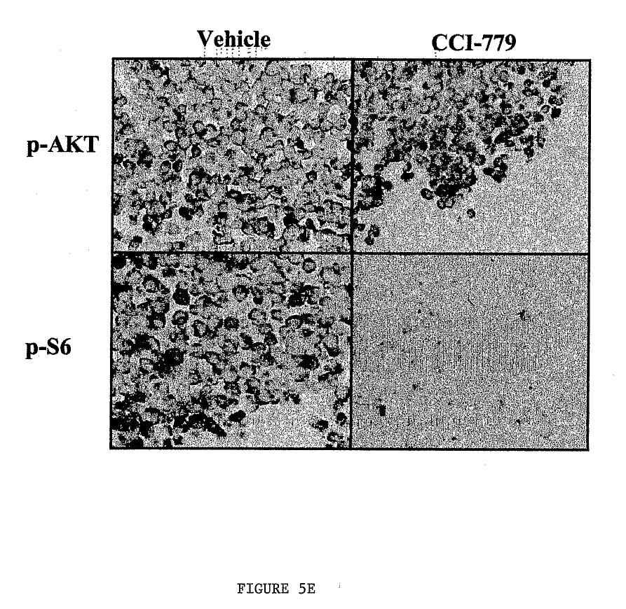

First, we characterized the specificity of the PTEN antibody used in our assays by probing cell lines with known endogenous PTEN expression. LNCaP cells, which have a deletion of one PTEN allele and a mutation of the other allele stained negatively while DU145 cells, which are PTEN wild, stained positively (FIG. 5A).

To validate the specificity of the phospho-specific antibodies, we used several confirmatory assays, i.e. preincubation with specific antigen peptides, immunofluorescence and treatment with pharmacological inhibitors of the pathway. We first examined the specificity of the antibodies when preincubated with a specific antigen peptide. Serial sections of a formalin fixed, paraffin embedded breast tumor were stained with p-AKT, p-S6, p-FKHR and p-ERK singly and then in combination with a specific antigen peptide. The phospho-specific staining was blocked when preincubated with its antigen peptide, confirming its specificity (FIG. 5B). Next, we examined the relationship between Total AKT and p-AKT by immunofluorescence. LNCaP cell pellets were probed with FITC labeled Total AKT (red) and p-AKT (green). LNCaP cells have loss of PTEN, with subsequent activation of AKT, as seen by the co-localization of AKT and p-AKT (yellow). When treated with the PI3K inhibitor LY294002, a decrease in the signal intensity was noted, consistent with known effects of this PI3K inhibitor (FIG. 5C). Next, we asked if LY294002 was able to block the activation of the signaling molecules downstream of PIP3. Addition of LY294002 successfully blocked the activation of AKT as well as S6, as seen by the decrease in staining intensity pre and post treatment. We noted that the staining intensity of p-FKHR in the untreated cells was weaker compared to AKT and S6 but that this was completely abolished by the addition of LY294002. Addition of the PI3K inhibitor appeared to have no effect on the staining intensity of p-ERK (FIG. 5D). We proceeded to examine the staining characteristics of p-ERK in HT129 cells, by inducing its expression by the addition of EGF alone (left panel), or in combination with a MEK inhibitor, UO-126. Addition of UO-126 to the induced cells resulted in decreased staining, consistent with in vitro signaling data (right panel).

Next we asked whether the phospho-specific antibodies could be used to monitor treatment effect. We probed LNCaP cells that had been treated with either CCI-779, an inhibitor of mTOR, with p-AKT and p-S6. As expected, pharmacological inhibition of mTOR resulted in downregulation of S6 phosphorylation but had no effect on p-AKT (FIG. 5E). This result raises the possibility of using these phospho-specific antibodies as tools to monitor target inhibition in patients treated with mTOR inhibitors.

Defining the Loss of PTEN Protein Expression In Vivo and Correlation with Signaling Pathway Upregulation

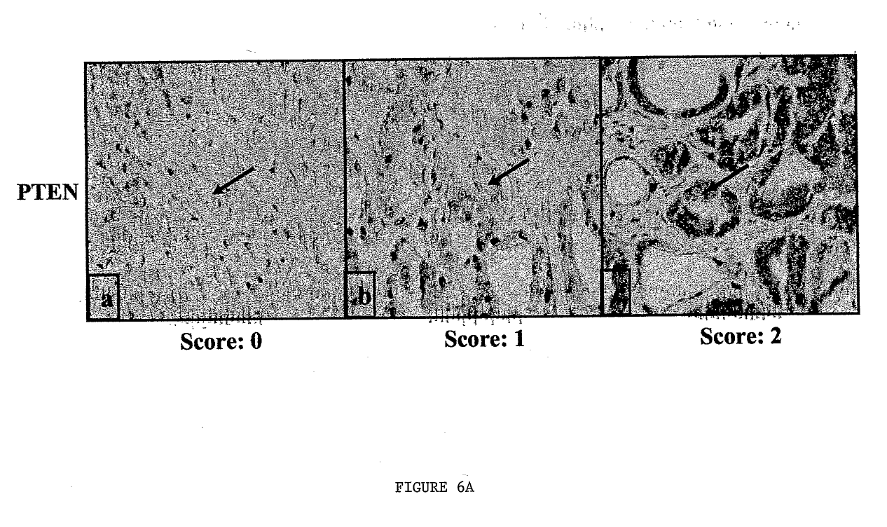

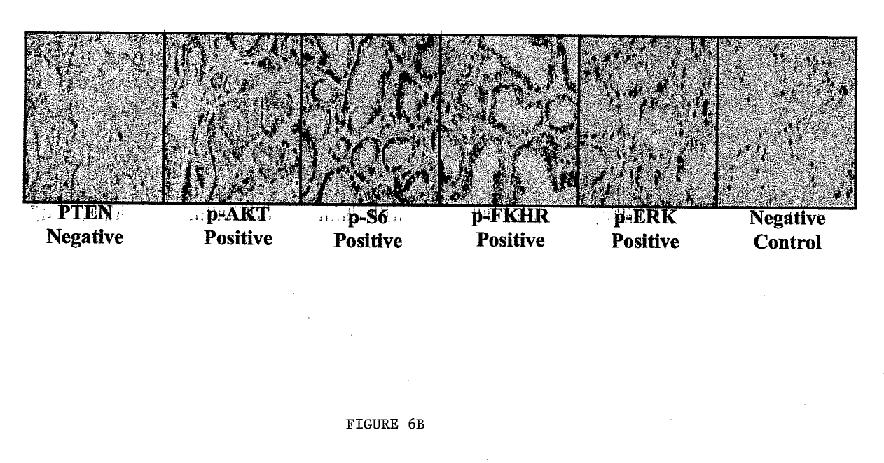

Having validated the performance characteristics of these antibodies in defined in vitro cell line systems, we next examined their immunohistochemical staining profiles within the tissue microarray. First, we defined the frequency of PTEN loss as measured by protein expression using an antibody to the C-terminus. [The C-terminal tail region contains PDZ domain binding sequences and multiple phosphorylation sites. We and others have shown that mutations in the C-terminal tail of PTEN results in loss of protein stability as well as decrease in membrane affinity (Wu et al., Proc Natl Acad Sci USA, 97: 4233-4238, 2000; Tortes et al., J Biol Chem, 276: 993-998, 2001; Das et al., Proc Natl Acad Sci USA, 100: 7491-7496, 2003). Loss of PTEN staining with this antibody should reflect impaired PTEN activity]. Since some cancers may have missense mutations in PTEN that do not alter protein expression, we recognize that this approach may underestimate the true frequency of PTEN loss and function. Of note, immunohistochemical assays will identify loss of protein expression in tumors with homozygous deletions, nonsense mutations, certain internal deletions, promoter methylation and finally posttranscriptional modifications. Complete loss of PTEN staining was seen in 29% (score=0; n=37); decreased staining compared to benign in 11% (Score=1; n=14), and staining intensity in tumor equivalent to that in benign glands in 60% (score=2; n=78; FIG. 6A). This is in keeping with previous reports of absent PTEN staining, performed on conventional tissue sections as well as tissue microarrays (McMenamin et al., Cancer Res, 59: 4291-4296, 1999; Halvorsen et al., Clin Cancer Res, 9: 1474-1479, 2003).

PTEN loss was significantly inversely correlated with activation of p-AKT (r=−0.46, P=0.00000002) but not p-ERK (FIG. 6B). Activation of p-AKT (Scheid et al., Mol Cell Biol, 22: 6247-6260, 2002) was significantly correlated with S6 phosphorylation (r=0.21, P=0.01) but not p-FKHR. Both AKT and S6 phosphorylation did not correlate with p-ERK, a target of the MAPK pathway (Table 7).

We next examined PTEN loss within a multivariate logistic regression model, with PTEN as the outcome and p-AKT, p-FKHR and p-S6 as covariates (Table 8). A strong independent association between PTEN and p-AKT was identified (P=0.0005), with the odds of a case being PTEN positive when p-AKT is positive is only 0.28 (95% CI, 0.14-0.58) that of a p-AKT negative case. Fitting the model for p-AKT and including p-FKHR and p-S6 as covariates demonstrated a significant independent association between AKT and p-S6 activation (P=0.028). The odds of a case being p-AKT positive when p-S6 is phosphorylated is 2.17 times (95% CI, 1.09-4.32) that of a case with p-S6 negative and AKT (positive vs. negative). The association between PTEN loss and p-S6 activation almost reached statistical significance (P=0.065).

We then asked if our data might be used to uncover relationships between these relationships without prior knowledge of the connectivity between these proteins using MDS analysis, a form of principal component analysis. MDS is an unsupervised data analysis method that allows examination of potential relationships between variables without assuming previous knowledge of their interaction. Using this approach, the signaling molecules are plotted in two dimensions and the distance between two molecules provide a measure of their inter-relatedness (Venables et al., Modern Applied Statistics with S-Plus. New York: Springer, 1999). This plot is a graphical representation of the correlations found in Table 7. Loss of PTEN protein expression and AKT activation are closely related, as shown by the short distance between them. In contrast, p-ERK is distant from PTEN and p-AKT (FIG. 7).

As shown above, utilizing prostate cancer tissue microarrays, we have successfully shown that this pathway can be interrogated in vivo, using immunohistochemical assays. We have identified a subset of patients who exhibit complete loss of PTEN protein expression. Absence of staining (i.e. negative staining), when viewed in conjunction with the appropriate controls is a firm, objective read-out and is readily reproducible across centers, analogous to the current standard of clinical care, where loss of basal cell marker expression (i.e. absent immunohistochemical staining for the high molecular weight keratin-903 and/or p63) is widely used as part of the diagnostic criteria to distinguish between benign as well as in-situ lesions and prostate cancer (Wojno et al., Am J Surg Pathol, 19: 251-260, 1995; Signoretti et al., Am J Pathol, 157: 1769-1775, 2000; Weinstein et al., Mod Pathol, 15: 1302-1308, 2002; Zhou et al., Am J Surg Pathol, 27: 365-371, 2003).

Loss of PTEN protein expression was significantly correlated with activation of AKT. Activation of AKT was in turn, associated with phosphorylation of S6, in keeping with what is known of the relationships between these signaling molecules (Luo et al., Cancer Cell, 4: 257-262, 2003; Schmelzle et al., Cell, 103: 253-262, 2000; Paez et al., Cancer Treat Res, 115: 145-167, 2003; Chou et al., Cell, 85: 573-583, 1996; Berven et al., Exp Cell Res, 296: 183-195, 2004). In terms of PTEN biology, it has been shown in genetic systems (i.e. conditional knock-outs) that the dosage of PTEN loss correlates with murine prostate cancer progression (Wang et al., Cancer Cell, 4: 209-221, 2003; Trotman et al., PLoS Biol, 1: E59, 2003). In our current series, PTEN loss, when examined by Pearson and Multivariate analysis, is significantly associated with phosphorylation of AKT. Conceptually, it may be that PTEN score 1 may reflect decreased (but not absent) gene activity.

The lack of correlation between p-AKT and p-FKHR requires further examination, especially in light of previously published work from out institution showing a significant association in glioblastomas (Choe et al., Cancer Res, 63: 2742-2746, 2003). When we were developing protocols to optimize the phospho-specific antibody immunohistochemical assays, we noted that the staining intensity of p-FKHR was less intense when compared to p-AKT or p-S6. This variation in staining characteristics, when transferred to human samples of different fixation and processing methodologies may lessen the likelihood of consistent, reproducible results. Of note, p-FKHR scored the lowest in the intra and inter-observer correlation percentiles, suggesting that subtle differences in staining intensity may lead to variations in the scoring protocol. p-ERK is not correlated with any molecules in this pathway, consistent with in vitro data that places the MAPK pathway parallel to, rather than epistatic to the PI3K cascade.

Using these phospho-specific antibodies, we were also able to follow changes in phosphorylation status of these proteins in a prostate cancer cell line, before and after treatment with CCI-779. This supports utility of these antibodies to confirm target inhibition in patient samples post treatment.

We did not see any correlation between PTEN status (or any of the other markets) and Gleason Score or the pathological stage. In contrast, two other studies have found associations between loss of PTEN protein expression or increased activation of AKT with higher Gleason scores and advanced stage (McMenamin et al., Cancer Res, 59: 4291-4296, 1999; Malik et al., Clin Cancer Res, 8: 1168-1171, 2002). One potential explanation is that our dataset may be underpowered to address this question, as it is populated by lower Gleason grades and pathologic stages. In future studies, it will be important to correlate PI3K pathway activation with clinical outcome.

From a clinical standpoint, it will be important to determine if the molecular aberrations seen in the tissue microarray cores can be reproduced in the context of a clinical trial where core needle biopsies might be used to guide treatment decisions with a targeted agent. Ultimately, these questions can only be accurately answered by examining the staining patterns in matched biopsy and radical prostatectomy specimens from the same patients, as we have done previously with other biomarkers such as p27 (Thomas et al., J Urol, 164: 1987-1991, 2000). Indeed, these results form the basis of multicenter, neoadjuvant clinical trial of an mTOR inhibitor in men with newly diagnosed, high-risk prostate cancer. Ultimately, it will be of interest to compare data such as this, obtained by immunohistochemical assays, with genomic data obtained by PTEN gene sequencing or LOH analysis from clinical material. At present, such techniques require that samples contain a high fraction of tumor cells (vs. normal) or isolation of pure populations of tumor cells by laser capture microdissection, thereby limiting their current use in the clinic.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

### Tables

TABLE 1
Patient Characteristics

| | |
|---|---|
| Clinical Characteristics | # |

Sex

| | |
|---|---|
| M | 29 |
| F | 16 |

Age (years)

| | |
|---|---|
| Median | 58 |
| Mean | 58 |
| Range | 28-88 |

Time to progression (days)

| | |
|---|---|
| Median | 183 |
| Mean | 227 |
| Range | 54-1006 |

Survival (days)

| | |
|---|---|
| Median | 412 |
| Mean | 427 |
| Range | 86-1794 |

TABLE 2
Association between PTEN expression and Akt pathway activation

| | | p-Akt | | p-mTOR | | p-FKHR | | | p-S6 | | p-ERK | | | |
|---|---|---|---|---|---|---|---|---|---|---|---|---|---|---|
| | 0 | 1 | 2 | 0 | 1 | 2 | 0 | 1 | 2 | 0 | 1 | 2 | + | − |
| PTEN− | 2 | 1 | 13 | 3 | 4 | 10 | 2 | 2 | 12 | 4 | 3 | 10 | 8 | 7 |
| PTEN+ | 18 | 3 | 4 | 6 | 8 | 11 | 10 | 2 | 13 | 5 | 7 | 12 | 16 | 8 |
| p-value | | | 0.00001 | | | ns | | | ns | | | ns | | ns |
| PTEN+/EGFR−/ | 6 | 2 | 0 | 5 | 2 | 0 | 7 | 0 | 1 | 5 | 2 | 0 | nd | nd |
| EGFRvIII− | | | | | | | | | | | | | | |
| PTEN+/EGFR+/ | 3 | 1 | 0 | 0 | 1 | 2 | 1 | 2 | 1 | 0 | 1 | 3 | nd | nd |
| EGFRvIII− | | | | | | | | | | | | | | |
| PTEN+/EGFR+/ | 7 | 0 | 4 | 0 | 3 | 9 | 2 | 1 | 10 | 0 | 3 | 10 | nd | nd |
| EGFRvIII+ | | | | | | | | | | | | | | |
| p-value | | | 0.06 | | | 0.001 | | | 0.002 | | | 0.001 | | nd |

TABLE 3
Association between Akt activation and downstream signaling

| | p- | p- | p- | | p- | p- | p- | | p- | p- | p- | |
|---|---|---|---|---|---|---|---|---|---|---|---|---|
| | mTOR | mTOR | mTOR | p- | FKHR | FKHR | FKHR | p- | S6 | S6 | S6 | p- |
| | 0 | 1 | 2 | value | 0 | 1 | 2 | value | 0 | 1 | 2 | value |
| p-Akt− | 6 | 7 | 7 | | 10 | 1 | 9 | | 6 | 7 | 7 | |
| p-Akt+ | 1 | 5 | 14 | 0.04 | 1 | 3 | 16 | 0.006 | 4 | 3 | 13 | 0.15 |
| | | | | | | | | | | | | (*0.003) |

TABLE 4
Univariate analysis between EGFR receptor
status and downstream signalling.

| | Pearson Correlation | p-value |
|---|---|---|
| EGFR | | |
| EGFRvIII | 0.31 | 0.04 |
| p-Erk | 0.34 | 0.03 |
| p-Akt | 0.07 | 0.67 |
| p-FKHR | 0.25 | 0.12 |
| p-mTOR | 0.24 | 0.13 |
| P-S6 | 0.3 | 0.06 |
| EGFRvIII | | |
| p-FKHR | 0.33 | 0.04 |
| p-mTOR | 0.31 | 0.06 |
| P-S6 | 0.3 | 0.06 |

TABLE 5
Univariate association between pathway activation and prognosis

| | Time to | | | |
|---|---|---|---|---|
| | progression | | Survival | |
| | (days) | p-value | (days) | p-value |
| p-Erk+ | 148 | | 356 | |
| p-Erk− | 263 | 0.05 | 488 | 0.02 |
| p-mTOR+/p-FKHR+/p-S6+ | 142 | 0.002 | 357 | 0.02 |
| p-mTOR−/p-FKHR−/p-S6− | 308 | | 528 | |
| p-Akt+ | 176 | NS | 358 | NS |
| p-Akt− | 238 | | 419 | |
| PTEN + | 153 | NS | 438 | NS |
| PTEN− | 212 | | 346 | |
| EGFR+ | 151 | NS | 385 | NS |
| EGFR− | 243 | | 412 | |
| EGFRvIII+ | 135 | NS | 420 | NS |
| EGFRvIII− | 230 | | 351 | |

TABLE 6

POLYPEPTIDE SEQUENCES
For convenience, Table 6 provides the sequences,
accession numbers and illustrative references for
the well known polypeptides discussed herein. In
certain sequences in this Table, illustrative
residues that are typically phosphorylated during
pathway signalling are shown in boldface type.

S6 (NP 001001, gi: 17158044) 249 amino acids
See, e.g. Pata et al., Gene 121 (2),
387-392 (1992)

(SEQ ID NO: 1)

MKLNISFPATGCQKLIEVDDERKLRTFYEKRMATEVAADALGEEWKGYVV
RISGGNDKQGFPMKQGVLTHGRVRLLLSKGHSCYRPRRTGERKRKSVRGC
IVDANLSVLNLVIVKKGEKDIPGLTDTTVPRRLGPKRASRIRKLFNLSKE
DDVRQYVVRKPLNKEGKKPRTKAPKIQRLVTPRVLQHKRRRIALKKQRTK
KNKEEAAEYAKLLAKRMKEAKEKRQEQIAKRRRLSSLRASTSKSESSQK
m-TOR (NP 004949, gi: 4826730) 2549 amino acids
See, e.g. Brown et al., Nature 369 (6483),
756-758 (1994)

(SEQ ID NO: 2)

MLGTGPAAATTAATTSSNVSVLQQFASGLKSRNEETRAKAAKELQHYVTM
ELREMSQEESTRFYDQLNHHIFELVSSSDANERKGGILAIASLIGVEGGN
ATRIGRFANYLRNLLPSNDPVVMEMASKAIGRLAMAGDTFTAEYVEFEVK
RALEWLGADRNEGRRHAAVLVLRELAISVPTFFFQQVQPFFDNIFVAVWD
PKQAIREGAVAALRACLILTTQREPKEMQKPQWYRHTFEEAEKGFDETLA
KEKGMNRDDRIHGALLILNELVRISSMEGERLREEMEEITQQQLVHDKYC
KDLMGFGTKPRHITPFTSFQAVQPQQSNALVGLLGYSSHQGLMGFGTSPS
PAKSTLVESRCCRDLMEEKFDQVCQWVLKCRNSKNSLIQMTILNLLPRLA
AFRPSAFTDTQYLQDTMNHVSCVKKEKERTAAFQALGLLSVAVRSEFKVY
LPRVLDIIRAALPPKDFAHKRQKAMQVDATVFTCISMLARAMGPGIQQDI
KELLEPMLAVGLSPALTAVLYDLSRQIPQLKKDIQDGLLKMLSLVLMHKP
LRHPGMPKGLAHQLASPGLTTLPEASDVGSITLALRTLGSFEFEGHSLTQ
FVRHCADHFLNSEHKEIRMEAARTCSRLLTPSIHLISGHAHVVSQTAVQV
VADVLSKLLVVGITDPDPDIRYCVLASLDERFDAHLAQAENLQALFVALN
DQVFEIRELAICTVGRLSSMNPAFVMPFLRKMLIQILTELEHSGIGRIKE
QSARMLGHLVSNAPRLIRPYMEPILKALILKLKDPDPDPNPGVINNVLAT
IGELAQVSGLEMRKWVDELFIIIMDMLQDSSLLAKRQVALWTLGQLVAST
GYVVEPYRKYPTLLEVLLNFLKTEQNQGTRREAIRVLGLLGALDPYKHKV
NIGMIDQSRDASAVSLSESKSSQDSSDYSTSEMLVNMGNLPLDEFYPAVS
MVALMRIFRDQSLSHHHTMVVQAITFIFKSLGLKCVQFLPQVMPTFLNVI
RVCDGAIREFLFQQLGMLVSFVKSHIRPYMDEIVTLMREFWVMNTSIQST
IILLIEQIVVALGGEFKLYLPQLIPHMLRVFMHDNSPGRIVSIKLLAAIQ
LFGANLDDYLHLLLPPIVKLFDAPEAPLPSRKAALETVDRLTESLDFTDY
ASRIIHPIVRTLDQSPELRSTAMDTLSSLVFQLGKKYQIFIPMVNKVLVR
HRINHQRYDVLICRIVKGYTLADEEEDPLIYQHRMLRSGQGDALASGPVE
TGPMKKLHVSTINLQKAWGAARRVSKDDWLEWLRRLSLELLKDSSSPSLR
SCWALAQAYNPMARDLFNAAFVSCWSELNEDQQDELIRSIELALTSQDIA
EVTQTLLNLAEFMEHSDKGPLPLRDDNGIVLLGERAAKCRAYAKALHYKE
LEFQKGPTPAILESLISINNKLQQPEAAAGVLEYAMKHFGELLEIQATWY
EKLHEWEDALVAYDKKMDTNKDDPELMLGRMRCLEALGEWGQLHQQCCEK
WTLVNDETQAKMARMAAAAAWGLGQWDSMEEYTCMIPRDTHDGAFYRAVL
ALHQDLFSLAQQCIDKARDLLDAELTAMAGESYSRAYGAMVSCHMLSELE
EVIQYKLVPERREIIRQIWWERLQGCQRIVEDWQKILMVRSLVVSPHEDM
RTWLKYASLCGKSGRLALAHKTLVLLLGVDPSRQLDHPLPTVHPQVTYAY
MKNMWKSARKIDAFQHMQHFVQTMQQQAQHAIATEDQQHKQELHKLMARC
FLKLGEWQLNLQGINESTIPKVLQYYSAATEHDRSWYKAWHAWAVMNFEA
VLHYKHQNQARDEKKKLRHASGANITNATTAATTAATATTTASTEGSNSE
SEAESTENSPTPSPLQKKVTEDLSKTLLMYTVPAVQGFFRSISLSRGNNL
QDTLRVLTLWFDYGHWPDVNEALVEGVKAIQIDTWLQVIPQLIARIDTPR
PLVGRLIHQLLTDIGRYHPQALIYPLTVASKSTTTARHNAANKILKNMCE
HSNTLVQQAMMVSEELIRVAILWHEMWHEGLEEASRLYFGERNVKGMFEV
LEPLHAMMERGPQTLKETSFNQAYGRDLMEAQEWCRKYMKSGNVKDLTQA
WDLYYHVFRRISKQLPQLTSLELQYVSPKLLMCRDLELAVPGTYDPNQPI
IRIQSIAPSLQVITSKQRPRKLTLMGSNGHEFVFLLKGHEDLRQDERVMQ
LFGLVNTLLANDPTSLRKNLSIQRYAVIPLSTNSGLIGWVPHCDTLHALI
RDYREKKKILLNIEHRIMLRMAPDYDHLTLMQKVEVFEHAVNNTAGDDLA
KLLWLKSPSSEVWFDRRTNYTRSLAVMSMVGYILGLGDRHPSNLMLDRLS
GKILHIDFGDCFEVAMTREKFPEKIPFRLTRMLTNAMEVTGLDGNYRITC
HTVMEVLREHKDSVMAVLEAFVYDPLLNWRLMDTNTKGNKRSRTRTDSYS
AGQSVEILDGVELGEPAHKKTGTTVPESIHSFIGDGLVKPEALNKKAIQI
INRVRDKLTGRDFSHDDTLDVPTQVELLIKQATSHENLCQCYIGWCPFW
FORKHEAD (NP 002006, gi: 9257222) 655 amino acids
See, e.g. Anderson et al., Genomics 47 (2),
187-199 (1998)

(SEQ ID NO: 3)

MAEAPQVVEIDPDFEPLPRPRSCTWPLPRPEFSQSNSATSSPAPSGSAAA
NPDAAAGLPSASAAAVSADFMSNLSLLEESEDFPQAPGSVAAAVAAAAAA
AATGGLCGDFQGPEAGCLHPAPPQPPPPGPLSQHPPVPPAAAGPLAGQPR
KSSSSRRNAWGNLSYADLITKAIESSAEKRLTLSQIYEWMVKSVPYFKDK
GDSNSSAGWKNSIRHNLSLHSKFIRVQNEGTGKSSWWMLNPEGGKSGKSP
RRRAASMDNNSKFAKSRSRAAKKKASLQSGQEGAGDSPGSQFSKWPASPG
SHSNDDFDNWSTFRPRTSSNASTISGRLSPIMTEQDDLGEGDVHSMVYPP
SAAKMASTLPSLSEISNPENMENLLDNLNLLSSPTSLTVSTQSSPGTMMQ
QTPCYSFAPPNTSLNSPSPNYQKYTYGQSSMSPLPQMPIQTLQDNKSSYG
GMSQYNCAPGLLKELLTSDSPPHNDIMTPVDPGVAQPNSRVLGQNVMMGP
NSVMSTYGSQASHNKMMNPSSHTHPGHAQQTSAVNGRPLPHTVSTMPHTS
GMNRLTQVKTPVQVPLPHPMQMSALGGYSSVSSCNGYGRMGLLHQEKLPS
DLDGMFIERLDCDMESIIRNDLMDGDTLDFNFDNVLPNQSFPHSVKTTTH
SWVSG
AKT (NP 005154 gi: 4885061) 480 amino acids
See, e.g. Staal, S.P., Proc. Natl. Acad. Sci.
U.S.A. 84 (14) , 5034-5037 (1987)

(SEQ ID NO: 4)

MSDVAIVKEGWLHKRGEYIKTWRPRYFLLKNDGTFIGYKERPQDVDQREA
PLNNFSVAQCQLMKTERPRPNTFIIRCLQWTTVIERTFHVETPEEREEWT
TAIQTVADGLKKQEEEEMDFRSGSPSDNSGAEEMEVSLAKPKHRVTMNEF
EYLKLLGKGTFGKVILVKEKATGRYYAMKILKKEVIVAKDEVAHTLTENR
VLQNSRHPFLTALKYSFQTHDRLCFVMEYANGGELFFHLSRERVFSEDRA
RFYGAEIVSALDYLHSEKNVVYRDLKLENLMLDKDGHIKITDFGLCKEGI
KDGATMKTFCGTPEYLAPEVLEDNDYGRAVDWWGLGVVMYEMMCGRLPFY
NQDHEKLFELILMEEIRFPRTLGPEAKSLLSGLLKKDPKQRLGGGSEDAK
EIMQHRFFAGIVWQHVYEKKLSPPFKPQVTSETDTRYFDEEFTAQMITIT
PPDQDDSMECVDSERRPHFPQFSYSASSTA
PTEN (NP 000305, gi: 4506249) 403 amino acids
See, e.g. Li et al., Science 275 (5308),
1943-1947 (1997)

(SEQ ID NO: 5)

MTAIIKEIVSRNKRRYQEDGFDLDLTYIYPNIIAMGFPAERLEGVYRNNI
DDVVRFLDSKHKNHYKIYNLCAERHYDTAKFNCRVAQYPFEDHNPPQLEL
IKPFCEDLDQWLSEDDNHVAAIHCKAGKGRTGVMICAYLLHRGKFLKAQE
ALDFYGEVRTRDKKGVTIPSQRRYVYYYSYLLKNHLDYRPVALLFHKMMF
ETIPMFSGGTCNPQFVVCQLKVKIYSSNSGPTRREDKFMYFEFPQPLPVC
GDIKVEFFHKQNKMLKKDKMFHFWVNTFFIPGPEETSEKVENGSLCDQEI
DSICSIERADNDKEYLVLTLTKNDLDKANKDKANRYFSPNFKVKLYFTKT
VEEPSNPEASSSTSVTPDVSDNEPDHYRYSDTTDSDPENEPFDEDQHTQI
TKV
FKHRL1 (O43524, gi: 8134467) 673 amino acids
See, e.g. Hillion et al., Blood.90 (9),
3714-3719 (1997)

(SEQ ID NO: 6)

MAEAPASPAPLSPLEVELDPEFEPQSRPRSCTWPLQRPELQASPAKPSGE
TAADSMIPEEEDDEDDEDGGGPAGSAMAIGGGGGSGTLGSGLLLEDSARV
LAPGGQDPGSGPATAAGGLSGGTQALLQPQQPLPPPQPGAAGGSGQPRKC
SSRRNAWGNLSYADLITRAIESSPDKRLTLSQIYEWMVRCVPYFKDKGDS
NSSAGWKNSIRHNLSLHSRFMRVQNEGTGKSSWWIINPDGGKSGKAPRRR
AVSMDNSNKYTKSRGRAAKKKAALQTAPESADDSPSQLSKWPGSPTSRSS
DELDAWTDFRSRTNSNASTVSGRLSPIMASTELDEVQDDDAPLSPMLYSS
SASLSPSVSKPCTVELPRLTDMAGTMNLNDGLTENLMDDLLDNITLPPSQ
PSPTGGLMQRSSSFPYTTKGSGLGSPTSSFNSTVFGPSSLNSLRQSPMQT
IQENKPATFSSMSHYGNQTLQDLLTSDSLSHSDVMMTQSDPLMSQASTAV
SAQNSRRNVMLRNDPMMSFAAQPNQGSLVNQNLLHHQHQTQGALGGSRAL
SNSVSNMGLSESSSLGSAKHQQQSPVSQSMQTLSDSLSGSSLYSTSANLP
VMGHEKFPSDLDLDMFNGSLECDMESIIRSELMDADGLDFNFDSLISTQN
VVGLNVGNFTGAKQASSQSWVPG
EGFR (NP 005219, gi: 29725609) 1210 amino acids
See, e.g. Tam et al., Nature 309 (5967),
418-425 (1984)

(SEQ ID NO: 7)

MRPSGTAGAALLALLAALCPASRALEEKKVCQGTSNKLTQLGTFEDHFLS
LQRMFNNCEVVLGNLEITYVQRNYDLSFLKTIQEVAGYVLIALNTVERIP
LENLQIIRGNMYYENSYALAVLSNYDANKTGLEKPMRNLQEILHGAVRFS
NNPALCNVESIQWRDDIVSSDFLSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLS
INATNIKHFKNCTSISGDLHILPVAFRGDSFTHTPPLDPQELDILKTVKE
ITGFLLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFMRRRHIVRKRTLRRLLQERELVEPLTPSGEAPN
QALLRILKEKETKKIKVLGSGAFGTVYKGLWIPEGEKVKIPVAIKELREA
TSPKANKEILDEAYVMASVDNPHVCRLLGICLTSTVQLITQMLPFGCLLD
YVREHKDNIGSQYLLNWCVQIAKGMNYLEDRRLVHRDLAARNVLVKTPQH
VKITDFGLAKLLGAEEKEYHAEGGKVPIKWMALESILHRIYTHQSDVWSY
GVTVWELMTFGSKPYDGIPASEISSILEKGERLPQPPICTIDVYMIMVKC
WMIDADSRPKFRELIIEFSKMARDPQRYLVIQGDERMHLPSPTDSNFYRA
MLDEEDMDDVVDADEYLIPQQGFFSSPSTSRTPLLSSLSATSNNSTVACI
DRNGLQSCPIKEDSFLQRYSSDPTGALTEDSIDDTFLPVPEYINQSVPKR
PAGSVQNPVYHNQPLNPAPSRDPHYQDPHSTAVGNPEYLNTVQPTCVNST
FDSPAHWAQKGSHQISLDNPDYQQDFFPKEAKPNGIFKGSTAENAEYLRV
APQSSEFIGA
pERK (XP 055766, gi: 20562757) 379 amino acids
See, e.g. Butch et al., J Biol Chem. 1996 Feb
23; 271 (8): 4230-5.

(SEQ ID NO: 8)

MAAAAAQGGGGGEPRRTEGVGPGVPGEVEMVKGQPFDVGPRYTQLQYIGE
GAYGMVSSAYDHVRKTRVAIKKISPFEHQTYCQRTLREIQILLRFRHENV
IGIRDILRASTLEAMRDVYIVQDLMETDLYKLLKSQQLSNDHUCYFLYQI
LRGLKYIHSANVLHRDLKPSNLLINTTCDLKICDFGLARIADPEHDHTGF
LTEYVATRWYRAPEIMLNSKGYTKSIDIWSVGCILAEMLSNRPIFPGKHY
LDQLNHILGILGSPSQEDLNCIINMKARNYLQSLPSKTKVAWAKLFPLSD
SKALDLLDRMLTFNPNKRITVEEALAHPYLEQYYDPTDEPVAEEPFTFAM
ELDDLPKERLKELIFQETARFQPGVLEAP
Ki-67 (CAA46519, gi: 415819) 3256 amino acids
See, e.g. Schluter et al., J. Cell Biol. 123 (3),
513-522 (1993)

(SEQ ID NO: 9)

MWPTRRLVTIKRSGVDGPHFPLSLSTCLFGRGIECDIRIQLPVVSKQHCK
IEIHEQEAILHNFSSTNPTQVNGSVIDEPVRLKHGDVITIIDRSFRYENE
SLQNGRKSTEFPRKIREQEPARRVSRSSFSSDPDEKAQDSKAYSKITEGK
VSGNPQVHIKNVKEDSTADDSKDSVAQGTTNVHSSEHAGRNGRNAADPIS
GDFKEISSVKLVSRYGELKSVPTTQCLDNSKKNESPFWKLYESVKKELDV
KSQKENVLQYCRKSGLQTDYATEKESADGLQGETQLLVSRKSRPKSGGSG
HAVAEPASPEQELDQNKGKGRDVESVQTPSKAVGASFPLYEPAKMKTPVQ
YSQQQNSPQKHKNKDLYTTGRRESVNLGKSEGFKAGDKTLTPRKLSTRNR
TPAKVEDAADSATKPENLSSKTRGSIPTDVEVLPTETEIHNEPFLTLWLT
QVERKIQKDSLSKPEKLGTTAGQMCSGLPGLSSVDINNFGDSINESEGIP
LKRRRVSFGGHLRPELFDENLPPNTPLKRGEAPTKRKSLVMHTPPVLKKI
IKEQPQPSGKQESGSEIHVEVKAQLSVISPPAPSPRKTPVASDQRRRSCK
TAPASSSKSQTEVPKRGGERVATCLQKRVSISRSQHDILQMICSKRRSGA
SEANLIVAKSWADVVKLGAKQTQTKVIKHGPQRSMNKRQRRPATPKKPVG
EVHSQFSTGHANSPCTIIIGKAHTEKVHVPARPYRVLNNFISNQKMDFKE
DLSGIAEMFKTPVKEQPQLTSTCHIAISNSENLLGKQFQGTDSGEEPLLP
TSESFGGNVFFSAQNAAKQPSDKCSASPPLRRQCIRENGNVAKTPRNTYK
MTSLETKTSDTETEPSKTVSTVNRSGRSTEFRNIQKLPVESKSEETNTEI
VECILKRGQKATLLQQRREGEMKEIERPFETYKENIELKENDEKMKAMKR
SRTWGQKCAPMSDLTDLKSLPDTELMKDTARGQNLLQTQDHAKAPKSEKG
KITKMPCQSLQPEPINTPTHTKQQLKASLGKVGVKEELLAVGKFTRTSGE
TTHTHREPAGDGKSIRTFKESPKQILDPAARVTGMKKWPRTPKEEAQSLE
DLAGFKELFQTPGPSEEMSTDEKTTKIACKSPPPESVDTPTITKQWPKRS
LRKADVEEEFLALRKLTPSAGKAMLTPKPAGGDEKDIDAFMGTPVQKLDL
AGTLPGSKRQLQTPKEKAQALEDLAGFKELFQTPGHTEELVAAGKTTKIP
CDSPQSDPVDTPTPTKQRPKRSIRKADVEGELLACRNLMPSAGKAMHTPK
PSVGEEKDIIIFVGTPVQKLKLTENLTGSKRRPQTPKEEAQALEDLTGFK
ELFQTPGHTEEAVAAGKTTKMPCESSPPESADTPTSTRRQPKTPLEKRDV
QKELSALKKLTQTSGETTHTDKVPGGEDKSINAFRETAKQKLDPAASVTG
SKRHPKTKEKAQPLEDLAGWKELFQTPVCTKDPTTHEKTTKIACRSQPDP
VDTPTSSKPQDKRSLRKVDVEEEFFALRKRTPSAGKAMHTPKPAVSGEKN
IYAFMGTPVQKLKLTENLTGSKRRLQTPKEKAQALEDLAGFKELFQTRGH
TEESMTNDKTAKVACKSSQPDLDKNPASSKRRLKTSLGKVGVKEELLAVG
LLTQTSGETTHTHTEPTGDGDSMKAFMESPKQILDSAASLTGSKRQLRTP
KGKSEVPEDLAGFIELFQTPSHTKESMTNEKTTKVSYRASQPDLVDTPTS
SKPQPKRSLRKADTEEEFLAFRKQTPSAGKAMHTPKPAVGEEKDINTFLG
TPVQKLDQPGNLPGSNRRLQTRKEKAQALEELTGFRELFQTPCTDNPTAD
EKTTKKILCKSPQSDPADTPTNTKQRPKRSLKKADVEEEFLAFRKLTPSA
GKAMHTPKAAVGEEKDINTFVGTPVEKLDLLGNLPGSK

TABLE 7
Univariate analysis

| | |
|---|---|
| Pearson | |
| Correlation | P |

PTEN

| | | |
|---|---|---|
| p-AKT | −0.46 | 0.00000002 |
| p-S6 | −0.16 | 0.07 |
| p-FKHR | −0.09 | 0.27 |
| p-ERK | −0.027 | 0.76 |

p-AKT

| | | |
|---|---|---|
| p-S6 | 0.21 | 0.01 |
| p-FKHR | −0.09 | 0.31 |
| p-ERK | −0.07 | 0.42 |

p-S6

| | | |
|---|---|---|
| p-FKHR | 0.04 | 0.59 |
| p-ERK | 0.16 | 0.09 |

TABLE 8
Multivariate logistic regression analysis

| Outcome | Covariates | Odds Ratio | 95% CI | P |
|---|---|---|---|---|
| PTEN | p-AKT | 0.28^{a} | (0.14-0.58) | 0.0005 |
| | p-S6 | 0.5 | (0.30-1.04) | 0.065 |
| | p-FKHR | 0.99 | (0.51-2.00) | 0.97 |
| p-AKT | p-S6 | 2.17 | (1.09-4.32) | 0.028 |
| | p-FKHR | 0.98 | (0.49-1.95) | 0.95 |

^{a}Odds of a tumor being PTEN deficient if it is p-AKT positive are 3.57 times (1/0.28) greater than if it is p-AKT negative

What is claimed is:

1. A method for identifying a mammalian tumor that is likely to respond, or is responsive to an EGFR polypeptide (SEQ ID NO: 7) inhibitor or an mTOR polypeptide (SEQ ID NO: 2) inhibitor, the method comprising examining a sample obtained from the tumor for:
(a) the expression of PTEN polypeptide (SEQ ID NO: 5);
and the presence of at least one of,
(b) phosphorylated S6 ribosomal polypeptide (SEQ ID NO: 1);
(c) EGFR polypeptide (SEQ ID NO: 7)
(d) phosphorylated AKT polypeptide (SEQ ID NO: 4); and
(e) phosphorylated ERK polypeptide (SEQ ID NO: 8)
wherein decreased expression of PTEN polypeptide together with decreased phosphorylation of S6 ribosomal polypeptide in the sample, as compared to a control, identifies the tumor as likely to respond or responsive to an mTOR inhibitor, and
wherein decreased expression an of PTEN together with normal phosphorylation of S6 ribosomal polypeptide in the sample, as compared to a control, identifies the tumor as not likely to respond or unresponsive to an mTOR inhibitor, and
wherein normal or increased expression of PTEN and increased expression and/or activity of EGFR together with increased phosphorylation of AKT and/or phosphorylation of ERK identifies the tumor as not likely to respond and/or unresponsive to an EGFR inhibitor.

2. The method of claim 1, wherein the mammalian tumor is a glioma or a cancer of the prostate, bile duct, bladder, breast, colon, endometrium, blood, liver, lung, skin, ovary, pancreas or thyroid.

3. The method of claim 1, wherein the phosphorylation of S6 ribosomal polypeptide is determined subsequent to contacting the tumor or sample with an mTOR inhibitor and/or the phosphorylation of AKT and/or ERK is determined subsequent to contacting the tumor or sample with an EGFR inhibitor.

4. The method of claim 1, wherein the mTOR inhibitor is rapamycin, SDZ-RAD, CCI-779, RAD 001, or AP23573.

5. The method of claim 1, wherein the EGFR inhibitor is ZD-1839, OSI-774, PD-153053, PD-168393, IMC-C225 or CI-1033.

6. The method of claim 1, wherein the expression of one or more of (a)-(e) is examined using an antibody.

7. The method of claim 6, wherein the presence of phosphorylated S6 ribosomal polypeptide (SEQ ID NO: 1) is examined using an antibody that binds an epitope comprising a phosphorylated serine residue at position 235 in SEQ ID NO: 1.

8. The method of claim 6, wherein the presence of EGFR and PTEN are examined using an EGFR-specific antibody and PTEN-specific antibody, respectively.

9. The method of claim 6, wherein the presence of phosphorylated AKT (SEQ ID NO: 4) is examined using an antibody that binds an epitope comprising a phosphorylated serine residue at position 473 in SEQ ID NO: 4.

10. The method of claim 6, wherein the presence of phosphorylated ERK is examined using an antibody that binds an epitope comprising a phosphorylated threonine residue at position 202 or a phosphorylated tyrosine residue at position 204 in SEQ ID NO: 8.

11. The method of claim 2, wherein the tumor is a glioblastoma multiforme tumor.

12. The method of claim 2, wherein the tumor is a prostate cancer.

13. The method of claim 1, wherein the sample is a paraffin embedded biopsy sample.

14. A method for identifying a mammalian tumor cell that does not express a PTEN polypeptide (SEQ ID NO: 5) and which is likely to respond or is responsive to an inhibitor of mTOR polypeptide (SEQ ID NO: 2) activity, the method comprising examining a sample obtained from the tumor for the presence of phosphorylated S6 ribosomal polypeptide (SEQ ID NO: 1) after contacting the tumor or the sample with the inhibitor,
wherein, an observable decrease in phosphorylated S6 ribosomal polypeptide in the sample, as compared to a control that is not contacted with the inhibitor identifies the tumor cells as likely to respond or responsive to the inhibitor, and
wherein no observable decrease in phosphorylated S6 ribosomal polypeptide in the sample, as compared to a control identifies the tumor cell as not likely to respond or unresponsive to the inhibitor.

15. The method of claim 14, wherein the tumor is glioma.

16. The method of claim 15, wherein the glioma is identified as tumor that does not express a PTEN polypeptide (SEQ ID NO: 5) by using an antibody that binds the PTEN polypeptide (SEQ ID NO: 5).

17. The method of claim 14, wherein the tumor is a prostate cancer.

18. A method for identifying a mammalian prostate tumor that expresses a PTEN polypeptide (SEQ ID NO: 5) and which is not likely to respond or is nonresponsive to an inhibitor of EGFR polypeptide (SEQ ID NO: 7) activity, the method comprising examining a sample obtained from the tumor for the presence of EGFR (SEQ ID NO: 7) and the presence of a phosphorylated AKT polypeptide (SEQ ID NO: 4) or the presence of a phosphorylated ERK polypeptide (SEQ ID NO: 8), after contacting the tumor or the sample with the inhibitor,
wherein an increase in the levels of the EGFR polypeptide and the levels of phosphorylated AKT polypeptide or phosphorylated ERK polypeptide identifies the prostate tumor as not likely to respond or nonresponsive to the inhibitor.

19. The method of claim 18, wherein the a sample obtained from the tumor is examined for the presence of a phosphorylated AKT polypeptide (SEQ ID NO: 4) and the presence of a phosphorylated ERK polypeptide (SEQ ID NO: 8).

20. The method of claim 18, wherein the tumor is obtained via biopsy.

21. The method of claim 18, wherein the tumor is identified as a tumor that expresses a PTEN polypeptide (SEQ ID NO: 5) using an antibody that binds the PTEN polypeptide (SEQ ID NO: 5).

22. A kit for characterizing a mammalian tumor or cell, the kit comprising:
(a) an antibody that binds PTEN (SEQ ID NO: 5);
and one or more of the following:
(b) an antibody that binds phosphorylated S6 ribosomal polypeptide (SEQ ID NO: 1);
(c) an antibody that binds EFGR (SEQ ID NO: 7);
(d) an antibody that binds phosphorylated AKT (SEQ ID NO: 4); and
(e) an antibody that binds phosphorylated ERK (SEQ ID NO: 8).

23. The kit of claim 22, wherein the kit comprises a plurality of antibodies selected from the group consisting of (b)-(e).

24. The kit of claim 22, wherein:
the antibody of (b) is specific for S6 ribosomal polypeptide (SEQ ID NO: 1) having a phosphorylated serine residue at position 235 in SEQ ID NO: 1;
the antibody of (d) is specific for AKT (SEQ ID NO: 4) having a phosphorylated serine residue at position 473 in SEQ ID NO: 4; and
the antibody of (e) is specific for ERK having a phosphorylated threonine residue at position 202 and tyrosine 204 in SEQ ID NO: 8.

25. The kit of claim 22, wherein the kit further includes an antibody that binds Ki-67 polypeptide (SEQ ID NO: 9), p-H3 histone polypeptide (SEQ ID NO: 10) or caspase-3 polypeptide (SEQ ID NO: 11).

26. The kit of claim 22, wherein the kit further includes; and at least one secondary antibody that binds to an antibody (a)-(e).
